(19) United States
(12) Patent Application Publication
(10) Pub. No.: US 2013/0042373 A1
(43) Pub. Date: Feb. 14, 2013

(54) CATION CHANNEL ACTIVITY

(71) Applicants: Mark Alfred Tester, Toorak Gardens (AU); Brent Kaiser, Eden Hills (AU); Scott Anthony William Carter, Novar Gardens (AU); Monique Shearer, Hawthorn (AU); Darren Craig Plett, Stepney (AU); Stuart John Roy, Glenelg East (AU); Olivier Cotsaftis, Fitzroy (AU); Stephan Tyerman, Urrbrae (AU); Mamoru Okamoto, Kingswood (AU)

(72) Inventors: Mark Alfred Tester, Toorak Gardens (AU); Brent Kaiser, Eden Hills (AU); Scott Anthony William Carter, Novar Gardens (AU); Monique Shearer, Hawthorn (AU); Darren Craig Plett, Stepney (AU); Stuart John Roy, Glenelg East (AU); Olivier Cotsaftis, Fitzroy (AU); Stephan Tyerman, Urrbrae (AU); Mamoru Okamoto, Kingswood (AU)

(21) Appl. No.: 13/498,133

(22) PCT Filed: Sep. 24, 2010
(86) PCT No.: PCT/AU2010/001256

§ 371 Date: Aug. 31, 2012

(30) Foreign Application Priority Data
Sep. 25, 2009 (AU) 2009904675

(51) Int. Cl. G01N 27/26; C12N 1/19; A01H 5/00; C12N 1/21; C12N 5/10; C12N 15/63; C12N 15/11; C12N 1/15

(52) U.S. Cl. 800/298; 536/23.1; 435/366; 435/349; 435/348; 435/419; 435/254.2; 435/254.11; 435/252.33; 435/412; 435/415; 435/411; 435/417; 435/375; 435/320.1; 435/414; 435/6.17; 435/325

(57) ABSTRACT

The present invention is predicated, in part, on the functional characterisation of membrane bound proteins that contribute to cation flux across biological membranes. In accordance with the present invention, it has been determined that at least some PQ-loop repeat polypeptides contribute to cation flux across biological membranes.

### PRIORITY CLAIM

This application claims priority to Australian provisional patent application 2009904675, filed 25 Sep. 2009, the content of which is hereby incorporated by reference.

### FIELD

The present invention relates to the functional characterisation of membrane bound proteins that contribute to cation flux across biological membranes.

### BACKGROUND

Voltage insensitive Non-Selective Cation Channels (viNSCCs) have been observed in many living systems, for example plants, animals and yeast. This class of cation channel is described as having a relatively low ability to discriminate between monovalent cations (eg. Na⁺, K⁺, NH₄⁺, Li⁺ etc) and being inhibited by divalent cations, such as Ca²⁺ and Mg²⁺.

Voltage insensitivity is not a strictly applied term. A protein showing some sensitivity to voltage may still be considered within the viNSCC class. As a result, although these proteins show some weak voltage dependence, they are still referred to as viNSCCs.

Extensive data has been generated about viNSCC proteins through the use of electrophysiology. viNSCCs are thought to be strong candidates behind observed low affinity cation fluxes in biological systems. These include the rapid flow of Na⁺ observed in plants exposed to saline soil conditions and the flux of NH₄⁺ into plants when their root systems are exposed to physiologically high NH₄⁺ concentrations.

Whereas salinity is a general term relating to all salts in the soil, the most relevant salt for a majority of cropping systems is NaCl. Salinity can impact the fitness of plants through effecting changes in the osmotic environment and through ionic toxicity. Primary flux of Na⁺ into the plant is facilitated by an unknown protein. Rapid accumulation of Na⁺ has been observed in plant shoots when roots have been exposed to saline conditions. Correlations have been drawn between Na⁺ accumulation in the shoots and Na⁺ toxicity symptoms.

This flux is a result of facilitated Na⁺ movement across cellular membranes. Whereas many proteins facilitating flow of Na⁺ across plant membranes have been described, the molecular identity of a protein that allows Na⁺ flux in the manner observed in Na⁺ accumulation experiments remains unknown.

Accordingly, identification and characterisation of membrane bound proteins that contribute to Na⁺, or other cation, flux across biological membranes would be desirable.

Reference to any prior art in this specification is not, and should not be taken as, an acknowledgment or any form of suggestion that this prior art forms part of the common general knowledge in any country.

### SUMMARY

The present invention is predicated, in part, on the functional characterisation of membrane bound proteins that contribute to cation flux across biological membranes.

Accordingly, in a first aspect, the present invention provides a method for modulating the rate, level or pattern of cation flux across a cell membrane, the method comprising modulating the expression of a PQ-loop repeat polypeptide in a cell comprising the cell membrane.

In some embodiments, the PQ loop repeat polypeptide comprises a YDR352w-like PQ loop repeat polypeptide.

In another embodiment, the PQ loop repeat polypeptide comprises a YOL092w-like PQ loop repeat polypeptide.

Generally, the PQ loop repeat polypeptides contemplated for use in accordance with the present invention include PQ loop repeat polypeptides which define a Voltage insensitive Non-Selective Cation Channel (viNSCC). In light of the above, in some embodiments, the PQ-loop repeat polypeptide comprises a monovalent cation transporter. In yet further embodiments, monovalent cation transport by the PQ loop repeat polypeptide may be inhibited by a polyvalent cation.

In some embodiments, modulating the rate, level or pattern of cation flux across a cell membrane of the cell modulates the cation tolerance or sensitivity of the cell. In one specific embodiment, the method may be used to increase the tolerance of a cell, such as a plant cell, to Na⁺ cations.

In a second aspect, the present invention provides a cell with a modulated rate, level or pattern of cation flux across a membrane of the cell, wherein said modulation is the result of modulated expression of a PQ-loop repeat polypeptide in the cell.

In some embodiments, the cell of the second aspect of the invention is produced according to a method of the first aspect of the invention.

In a third aspect, the present invention provides a multicellular structure comprising one or more cells of the second aspect of the invention.

In a fourth aspect, the present invention provides a nucleic acid construct or vector comprising a PQL nucleic acid. In some embodiments, the construct is an expression construct which may be used to effect expression of a PQ loop repeat polypeptide in a cell as described with reference to the first aspect of the invention.

In a fifth aspect, the present invention provides a genetically modified cell comprising a nucleic acid construct or vector of the fourth aspect of the invention.

Furthermore, in a sixth aspect, the present invention provides a multicellular structure comprising one or more cells of the fifth aspect of the invention.

In a seventh aspect, the present invention provides a method for ascertaining the cation sensitivity or tolerance of an organism, the method comprising determining the expression of a PQ loop repeat polypeptide in one or more cells of the organism. In some embodiments, a relatively high level of expression is associated with cation sensitivity in the organism. In another embodiment, a relatively low level of expression is associated with cation tolerance in the organism.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to herein by a sequence identifier number (SEQ ID NO:). The SEQ ID NOs: correspond numerically to the sequence identifiers <400> 1 (SEQ ID NO:1), <400> 2 (SEQ ID NO: 2), etc. A summary of the sequence identifiers is provided in Table 1. A sequence listing is provided at the end of the specification.

TABLE 1
Summary of Sequence Identifiers

| Sequence | |
|---|---|
| Identifier | Sequence |
| SEQ ID NO: 1 | RLPQIXXN amino acid motif |
| SEQ ID NO: 2 | YXLS amino acid motif |
| SEQ ID NO: 3 | YDR352w-like polypeptide amino acid consensus |
| | sequence |
| SEQ ID NO: 4 | YDR352w polypeptide amino acid sequence |
| SEQ ID NO: 5 | PQIXXNF amino acid motif |
| SEQ ID NO: 6 | GDIFNL amino acid motif |
| SEQ ID NO: 7 | PQIXLNXKR amino acid motif |
| SEQ ID NO: 8 | YOL092w-like polypeptide amino acid consensus |
| | sequence |
| SEQ ID NO: 9 | YOL092w polypeptidc amino acid sequence |
| SEQ ID NO: 10 | AtPQL1 forward primer nucleotide sequence |
| SEQ ID NO: 11 | AtPQL1 reverse primer nucleotide sequence |
| SEQ ID NO: 12 | AtPQL2 forward primer nucleotide sequence |
| SEQ ID NO: 13 | AtPQL2 reverse primer nucleotide sequence |
| SEQ ID NO: 14 | AtPQL3 forward primer nucleotide sequence |
| SEQ ID NO: 15 | AtPQL3 reverse primer nucleotide sequence |
| SEQ ID NO: 16 | OsPQL1 forward primer nucleotide sequence |
| SEQ ID NO: 17 | OsPQL1 reverse primer nucleotide sequence |
| SEQ ID NO: 18 | I miR-s primer nucleotide sequence |
| SEQ ID NO: 19 | IImiR-a primer nucleotide sequence |
| SEQ ID NO: 20 | III miR*s primer nucleotide sequence |
| SEQ ID NO: 21 | IV miR*a primer nucleotide sequence |
| SEQ ID NO: 22 | I miR-s primer nucleotide sequence |
| SEQ ID NO: 23 | IImiR-a primer nucleotide sequence |
| SEQ ID NO: 24 | III miR*s primer nucleotide sequence |
| SEQ ID NO: 25 | IV miR*a primer nucleotide sequence |
| SEQ ID NO: 26 | I miR-s primer nucleotide sequence |
| SEQ ID NO: 27 | IImiR-a primer nucleotide sequence |
| SEQ ID NO: 28 | III miR*s primer nucleotide sequence |
| SEQ ID NO: 29 | IV miR*a primer nucleotide sequence |
| SEQ ID NO: 30 | I miR-s primer nucleotide sequence |
| SEQ ID NO: 31 | IImiR-a primer nucleotide sequence |
| SEQ ID NO: 32 | III miR*s primer nucleotide sequence |
| SEQ ID NO: 33 | IV miR*a primer nucleotide sequence |
| SEQ ID NO: 34 | I miR-s primer nucleotide sequence |
| SEQ ID NO: 35 | IImiR-a primer nucleotide sequence |
| SEQ ID NO: 36 | III miR*s primer nucleotide sequence |
| SEQ ID NO: 37 | IV miR*a primer nucleotide sequence |
| SEQ ID NO: 38 | I miR-s primer nucleotide sequence |
| SEQ ID NO: 39 | IImiR-a primer nucleotide sequence |
| SEQ ID NO: 40 | III miR*s primer nucleotide sequence |
| SEQ ID NO: 41 | IV miR*a primer nucleotide sequence |
| SEQ ID NO: 42 | I miR-s primer nucleotide sequence |
| SEQ ID NO: 43 | II miR-a primer nucleotide sequence |
| SEQ ID NO: 44 | III miR*s primer nucleotide sequence |
| SEQ ID NO: 45 | IV miR*a primer nucleotide sequence |
| SEQ ID NO: 46 | I miR-s primer nucleotide sequence |
| SEQ ID NO: 47 | IImiR-a primer nucleotide sequence |
| SEQ ID NO: 48 | III miR*s primer nucleotide sequence |
| SEQ ID NO: 49 | IV miR*a primer nucleotide sequence |
| SEQ ID NO: 50 | amiRNA AtPQL1 [symbol] 2 #1 target nucleotide sequence |
| SEQ ID NO: 51 | amiRNA AtPQL1 [symbol] 2 #2 target nucleotide sequence |
| SEQ ID NO: 52 | amiRNA AtPQL1 [symbol] 3 #1 target nucleotide sequence |
| SEQ ID NO: 53 | amiRNA AtPQL1 [symbol] 3 #2 target nucleotide sequence |
| SEQ ID NO: 54 | amiRNA AtPQL2 [symbol] 3 #1 target nucleotide sequence |
| SEQ ID NO: 55 | amiRNA AtPQL2 [symbol] 3 #2 target nucleotide sequence |
| SEQ ID NO: 56 | amiRNA AtPQL1, 2 [symbol] 3 #1 target nucleotide |
| | sequence |
| SEQ ID NO: 57 | amiRNA AtPQL1, 2 [symbol] 3 #2 target nucleotide |
| | sequence |

### DESCRIPTION OF EXEMPLARY EMBODIMENTS

It is to be understood that the following description is for the purpose of describing particular embodiments only and is not intended to be limiting with respect to the above description.

As set out above, the present invention is predicated, in part, on the functional characterisation of membrane bound proteins that contribute to cation flux across biological membranes. In accordance with the present invention, it has been determined that at least some PQ-loop repeat polypeptides contribute to cation flux across biological membranes.

Accordingly, in a first aspect, the present invention provides a method for modulating the rate, level or pattern of cation flux across a cell membrane, the method comprising modulating the expression of a PQ-loop repeat polypeptide in a cell comprising the cell membrane.

“PQ loop repeat polypeptides”, as contemplated herein, are characterised by repeats of a proline and glutamine (PQ) residues prior to an extra membrane loop. This motif is referred to herein as a “PQ loop motif”. Generally, a PQ loop repeat polypeptide comprises 1, 2, 3, 4 or 5 PQ loop repeat motifs. In some embodiments, the term PQ loop repeat polypeptide should be understood to include a polypeptide comprising one or two PQ loop motifs.

In some embodiments, the PQ loop repeat polypeptide comprises a YOL092w-like PQ loop repeat polypeptide.

In some embodiments, a YOL092w-like PQ loop repeat polypeptide may comprise one or more amino acid motifs selected from the list consisting of:

(SEQ ID NO: 5)
PQIXXNF

wherein residue 3 (I) may be replaced with L or V, residues 4 and 5 (X) may each be any amino acid residue or either or both may be absent, and residue 7 (F) may be replaced with Y;
or

(SEQ ID NO: 6)
GDIFNL

wherein residue 3 (I) may be replaced with V or F, and residue 6 (L) may be replaced with V;
or

(SEQ ID NO: 7)
PQIXLNXKR

Wherein residue 3 (I) may be replaced with A, residue 4 (X) may be any amino acid residue or may be absent, reside 5 (L) may be replaced with K or M, residue 7 (X) may be any amino acid residue or may be absent, residue 8 (K) may be replaced with R and residue 9 (R) may be replaced with A.

In some embodiments, a YOL092w-like PQ loop repeat polypeptide may comprise:

the amino acid motifs set forth in SEQ ID NO: 5 and SEQ ID NO: 6;
the amino acid motifs set forth in SEQ ID NO: 5 and SEQ ID NO: 7;
the amino acid motifs set forth in SEQ ID NO: 6 and SEQ ID NO: 7; or
the amino acid motifs set forth in SEQ ID NO: 5, SEQ ID NO: 6 and SEQ ID NO: 7.

In further embodiments, a YOL092w-like PQ loop repeat polypeptide may comprise an amino acid which is at least 20% identical to the YOL092w-like PQ loop repeat polypeptide consensus sequence set forth in SEQ ID NO: 8.

In a yet further embodiment, a YOL092w-like PQ loop repeat polypeptide may comprise an amino acid sequence which is at least 20% identical to the amino acid sequence of the YOL092w PQ loop repeat polypeptide set forth in SEQ ID NO: 9.

Reference herein to “at least 20% identity” should be understood to also include levels of identity higher than at least 20% including, for example, at least 30% identity, at least 40% identity, at least 50% identity, at least 60% identity, at least 70% identity, at least 80% identity, at least 90% identity or at least 95% identity.

When comparing amino acid sequences, the compared sequences should be compared over a comparison window of at least 50 amino acid residues, at least 100 amino acid residues, at least 200 amino acid residues, at least 300 amino acid residues or over the full length of SEQ ID NO: 8 or SEQ ID NO: 9. The comparison window may comprise additions or deletions (ie. gaps) of about 20% or less as compared to the reference sequence (which does not comprise additions or deletions) for optimal alignment of the two sequences. Optimal alignment of sequences for aligning a comparison window may be conducted by computerized implementations of algorithms such the BLAST family of programs as, for example, disclosed by Altschul et al. (Nucl. Acids Res. 25: 3389-3402, 1997). A detailed discussion of sequence analysis can be found in Unit 19. 3 of Ausubel et al. (“Current Protocols in Molecular Biology” John Wiley & Sons Inc, 1994-1998, Chapter 15, 1998).

The term “YOL092w-like PQ loop repeat polypeptide” also includes “functional homologs” of a polypeptide comprising the amino acid sequence set forth in SEQ ID NO: 9. Functional homologs include any PQ loop repeat polypeptide which is able to modulate the rate, level or pattern of cation flux across a cell membrane.

Notwithstanding the above, the functional homolog may comprise, for example, a polypeptide which has one or more amino acid insertions, deletions or substitutions relative to the polypeptide comprising the amino acid sequence set forth in SEQ ID NO: 9; a mutant form or allelic variant of the polypeptide comprising the amino acid sequence set forth in SEQ ID NO: 9; an ortholog of the polypeptide comprising the amino acid sequence set forth in SEQ ID NO: 9; an analog of the polypeptide comprising the amino acid sequence set forth in SEQ ID NO: 9; and the like.

Examples of YOL092w-like PQ loop repeat polypeptides include polypeptides having the following accession numbers: NP_{—}014549 (Saccharomyces cerevisiae YOL092w), EDN64758 (Saccharomyces cerevisiae YJM789), NP_{—}009705 (Saccharomyces cerevisiae YBR147w), NP_{—}594596 (Stm1 Shizosacharomyces pombe), Arabidopsis thaliana At4g20100, Arabidopsis thaliana At4g36850, AAK76703 (Arabidopsis thaliana), Arabidopsis thaliana At2g41050, Arabidopsis thaliana At5g59470, Arabidopsis thaliana At5g40670, Arabidopsis thaliana At4g07390, Oryza sativa Os01 g16170, Oryza sativa Os07g29610, Oryza sativa Os12g18110, Oryza sativa Os01g0266800, XP_{—}001753587 (Physcomitrella patens), Physcomitrella patens Pp174957, Physcomitrella patens Pp182799, Physcomitrella patens Pp217317 and Physcomitrella patens Pp210671

In some embodiments, the YOL092w-like PQ loop repeat polypeptide comprises at least 5 transmembrane domains. In some embodiments, the YOL092w-like PQ loop repeat polypeptide comprises at least 7 transmembrane domains. In further embodiments, the YOL092w-like PQ loop repeat polypeptide comprises 7 transmembrane domains together with a cytoplasmic loop connecting transmembrane domains 3 and 4.

In some embodiments, the PQ loop repeat polypeptide comprises a YDR352w-like PQ loop repeat polypeptide.

In some embodiments, a YDR352w-like PQ loop repeat polypeptide may comprise one or more amino acid motifs selected from the list consisting of:

(SEQ ID NO: 1)
RLPQIXXN

wherein residue 2 (L) may be replaced with I, residue 5 (I) may be replaced with V, F or L, residues 6 and 7 (X) may each be any amino acid residue or either or both may be absent, and residue 8 (N) may be replaced with C or I;
or

(SEQ ID NO: 2)
YXLS

wherein residue 1 (Y) may be replaced with R, residue 2 (X) may be any amino acid residue or may be absent, residue 3 (L) may be replaced with F or I and residue 4 (S) may be replaced with T.

In some embodiments, a YDR352w-like PQ loop repeat polypeptide may comprise each of the amino acid motifs set forth in SEQ ID NO: 1 and SEQ ID NO: 2.

In further embodiments, a YDR352w-like PQ loop repeat polypeptide may comprise an amino acid which is at least 20% identical to the YDR352w-like PQ loop repeat polypeptide consensus sequence set forth in SEQ ID NO: 3.

In a yet further embodiment, a YDR352w-like PQ loop repeat polypeptide may comprise an amino acid sequence which is at least 20% identical to the amino acid sequence of the YDR352w PQ loop repeat polypeptide set forth in SEQ ID NO: 4.

Reference herein to “at least 20% identity” should be understood to also include levels of identity higher than at least 20% including, for example, at least 30% identity, at least 40% identity, at least 50% identity, at least 60% identity, at least 70% identity, at least 80% identity, at least 90% identity or at least 95% identity.

When comparing amino acid sequences, the compared sequences should be compared over a comparison window of at least 50 amino acid residues, at least 100 amino acid residues, at least 200 amino acid residues, at least 300 amino acid residues or over the full length of SEQ ID NO: 3 or SEQ ID NO: 4. The comparison window may comprise additions or deletions (ie. gaps) of about 20% or less as compared to the reference sequence (which does not comprise additions or deletions) for optimal alignment of the two sequences. Optimal alignment of sequences for aligning a comparison window may be conducted by computerized implementations of algorithms such the BLAST family of programs as, for example, disclosed by Altschul et al. (Nucl. Acids Res. 25: 3389-3402, 1997). A detailed discussion of sequence analysis can be found in Unit 19. 3 of Ausubel et al. (“Current Protocols in Molecular Biology” John Wiley & Sons Inc, 1994-1998, Chapter 15, 1998).

The term “YDR352w-like PQ loop repeat polypeptide” also includes “functional homologs” of a polypeptide comprising the amino acid sequence set forth in SEQ ID NO: 4. Functional homologs include any PQ loop repeat polypeptide which is able to modulate the rate, level or pattern of cation flux across a cell membrane.

Notwithstanding the above, the functional homolog may comprise, for example, a polypeptide which has one or more amino acid insertions, deletions or substitutions relative to the polypeptide comprising the amino acid sequence set forth in SEQ ID NO: 4; a mutant form or allelic variant of the polypeptide comprising the amino acid sequence set forth in SEQ ID NO: 4; an ortholog of the polypeptide comprising the amino acid sequence set forth in SEQ ID NO: 4; an analog of the polypeptide comprising the amino acid sequence set forth in SEQ ID NO: 4; and the like.

Examples of YDR352w-like PQ loop repeat polypeptides include polypeptides having the following accession numbers: gi|151942326 Saccharomyces cerevisiae YJM789, gi|167389211 Saccharomyces cerevisiae YDR352w, gi|114554372 Pan troglodytes, gi|92110021 Homo sapiens, gi|34526924 Homo sapiens, gi|168002094 Physcomitrella patens, gi|119615288 HOMO sapiens, gi|153791811 Homo sapiens, gi|119599108 Homo sapiens, gi|47077705 Homo sapiens, and Os7g29610.

Generally, the PQ loop repeat polypeptides contemplated for use in accordance with the present invention include PQ loop repeat polypeptides which define a Voltage insensitive Non-Selective Cation Channel (viNSCC).

As referred to herein, a “Voltage insensitive Non-Selective Cation Channel” or “viNSCC” refers to a cation channel polypeptide having a relatively low ability to discriminate between monovalent cations (eg. Na⁺, K⁺, NH₄⁺, Li⁺). In some embodiments viNSCCs may also be inhibited by polyvalent cations, such as Ca²⁺ and Mg²⁺. The term “voltage insensitivity” is not a strictly applied term when used with reference to viNSCCs. As such, a polypeptide showing some sensitivity to voltage may still be considered within the viNSCC class (for example, see Davenport and Tester, Plant Physiol. 122: 823-834, 2000). Thus, although a PQ-loop repeat polypeptide may show some weak voltage dependence, it may still be considered a viNSCC within the scope of the present invention. In accordance with the above, viNSCCs may also be referred to as NSCCs, and for the purposes of this specification, the two terms should be considered synonymous and interchangeable.

In light of the above, in some embodiments, the PQ-loop repeat polypeptide comprises a monovalent cation transporter. In further embodiments, the monovalent cation comprises one or more of Na⁺, NH₄⁺, methylammonium, Tris⁺ or choline⁺.

In yet further embodiments, monovalent cation transport by the PQ loop repeat polypeptide may be inhibited by a polyvalent cation.

Examples of polyvalent cations that may inhibit monovalent cation transport by the PQ loop repeat protein include divalent cations including Be²⁺, Mg²⁺, Ca²⁺, Sr²⁺, Ba²⁺ and Ra²⁺ as well as divalent transition metal ions such as Zn²⁺, Fe²⁺ and the like. In some embodiments, the divalent cation may be Ca²⁺.

In some embodiments, the polyvalent cation may be a trivalent cation such as La³⁺.

As set out above, the present invention provides a method for modulating the rate, level or pattern of cation flux across a cell membrane.

As referred to herein, a “cell membrane” may be any membrane of a cell across which it may be desirable to modulate the rate, level or pattern or cation flux. Examples of such cell membranes include, for example, the plasma membrane or organelle membranes such as chloroplast membranes, thylakoid membranes, mitochondrial membranes (inner or outer), endoplasmic reticulum, golgi apparatus membranes, vacuolar membranes, nuclear membranes, acrosome membranes, autophagosome membranes, glycosome membranes, glyoxysome membranes, hydrogenosome membranes, lysosome membranes, melanosome membranes, mitosome membranes, peroxisome membranes, vesicle membranes, and the like.

In some embodiments, the present invention provides a method for modulating the rate, level or pattern of cation flux across a cell plasma membrane.

The cells contemplated by the present invention may include any cell comprising a membrane as discussed above. As such, the cell may be an animal cell including a mammalian cell, a human cell, a bird cell, an insect cell, a reptile cell and the like; a plant cell including angiosperm or gymnosperm higher plants as well as lower plants such as bryophytes, ferns and horsetails; a fungal cell such as a yeast or filamentous fungus and the like. Alternatively, the cell may also be a prokaryotic cell such as a bacterial cell (eg. an E. coli cell), or an archaea cell.

In some embodiments, the cell may be, for example, a plant cell, a vascular plant cell, including a monocotyledonous or dicotyledonous angiosperm plant cell or a gymnosperm plant cell. In some embodiments, the plant cell is a monocotyledonous plant cell. In some embodiments, the monocotyledonous plant cell may be a cereal crop plant cell.

As used herein, the term “cereal crop plant” includes members of the Poaceae (grass family) that produce edible grain for human or animal food. Examples of Poaceae cereal crop plants include barley, wheat, rice, maize, millets, sorghum, rye, triticale, oats, teff, wild rice, spelt and the like. However, the term cereal crop plant should also be understood to include a number of non-Poaceae species that also produce edible grain and are known as the pseudocereals, such as amaranth, buckwheat and quinoa.

In further embodiments, the plant may be a dicotyledonous plant including, for example, legumes such as soybeans (Glycine spp.), peas and clovers, other dicotyledonous oil-seed crops such as Brassica spp. and solanaceous crop plants such as tomato, pepper, chilli, potato, eggplant and the like.

In some embodiments, modulating the rate, level or pattern of cation flux across a cell membrane of the cell modulates the cation tolerance or sensitivity of the cell.

For example, in some embodiments, a PQ loop repeat polypeptide in a cell may be downregulated in order to reduce flux of a particular cation across the plasma membrane and thus either reduce the sensitivity of the cell to the cation and/or increase the tolerance of the cell to environmental cations. In some embodiments the cation may be a monovalent cation. In further embodiments the cation may comprise any one or more of Na⁺, K⁺, NH₄⁺, methylammonium, Tris⁺, or choline⁺. In some embodiments, the method may be used to increase the tolerance of a cell, such as a plant cell, to Na⁺ cations.

Conversely, the present invention also contemplates increasing the flux of a cation across a cell membrane. In these embodiments, the expression of a PQ loop repeat polypeptide may be increased in a cell. As described later in the examples, increasing membrane flux of a cation constitutively in all cells of an organism may increase the tolerance of the organism as a whole to the cation.

As set out above, the present invention is predicated, in part, on modulating the expression of a PQ loop repeat polypeptide in a cell.

As referred to herein, modulation of the “expression” of a PQ loop repeat polypeptide includes modulating the level and/or activity of the polypeptide.

Modulation of the “level” of the polypeptide should be understood to include an increase or decrease in the level or amount of a PQ loop repeat polypeptide in a cell or a particular part of a cell. Similarly, modulation of the “activity” of a PQ loop repeat polypeptide should be understood to include an increase or decrease in, for example, the total activity, specific activity, half-life and/or stability of a PQ loop repeat polypeptide in the cell.

By “increasing” is intended, for example, a 1%, 5%, 10%, 20%, 30%, 40%, 50%, 60%, 70%, 80%, 90%, 100%, 2-fold, 5-fold, 10-fold, 20 fold, 50-fold, 100-fold increase in the level of activity of a PQ loop repeat polypeptide in the cell. By “decreasing” is intended, for example, a 1%, 5%, 10%, 20%, 30%, 40%, 50%, 60%, 70%, 80%, 90%, 91%, 92%, 93%, 94%, 95%, 96%, 97%, 98%, 99%, 100% reduction in the level or activity of a PQ loop repeat polypeptide in the cell.

“Modulating” should also be understood to include introducing a particular PQ loop repeat polypeptide into a cell which does not normally express the introduced polypeptide, or the substantially complete inhibition of a PQ loop repeat polypeptide activity in a cell that normally expresses such a polypeptide.

The present invention contemplates any means by which the expression of a PQ loop repeat polypeptide in a cell may be modulated. This includes, for example, methods such as the application of agents which modulate PQ loop repeat polypeptide activity in a cell, including the application of agonists or antagonists; the application of agents which mimic PQ loop repeat polypeptide activity in a cell; modulating the expression of a nucleic acid which encodes a PQ loop repeat polypeptide in the cell; effecting the expression of an altered or mutated nucleic acid in a cell such that a PQ loop repeat polypeptide with increased or decreased specific activity, half-life and/or stability is expressed by the cell; or modulating the expression pattern and/or targeting of a PQ loop repeat polypeptide in a cell for example via modification of a transcriptional control sequence and/or signal polypeptide associated with the PQ loop repeat polypeptide.

In some embodiments, the expression of the polypeptide is modulated by modulating the expression of a nucleic acid which encodes a PQ loop repeat polypeptide in the cell.

As referred to herein, a nucleic acid which encodes a PQ loop repeat polypeptide (“PQL nucleic acid”) refers to any nucleic acid which encodes a PQ loop repeat polypeptide or a functional active fragment or variant of such a polypeptide. Specific examples of PQL nucleic acids include nucleic acids which encode the PQ loop repeat polyepeptides hereinbefore described.

The PQL nucleic acids of the present invention may be derived from any source. For example, the PQL nucleic acids may be derived from an organism, such as a plant, animal or fungus. Alternatively, the PQL nucleic acid may be a synthetic nucleic acid.

The PQL nucleic acids contemplated by the present invention may also comprise one or more non-translated regions such as 3′ and 5′ untranslated regions and/or introns.

The PQL nucleic acids contemplated by the present invention may comprise, for example, mRNA sequences, cDNA sequences or genomic nucleotide sequences.

The term “modulating” with regard to the expression of a PQL nucleic acid may include increasing or decreasing the transcription and/or translation of a PQL nucleic acid in a cell.

By “increasing” is intended, for example a 1%, 5%, 10%, 20%, 30%, 40%, 50%, 60%, 70%, 80%, 90%, 100%, 2-fold, 5-fold, 10-fold, 20-fold, 50-fold, 100-fold or greater increase in the transcription and/or translation of a PQL nucleic acid. By “decreasing” is intended, for example, a 1%, 5%, 10%, 20%, 30%, 40%, 50%, 60%, 70%, 80%, 90%, 91%, 92%, 93%, 94%, 95%, 96%, 97%, 98%, 99%, 100% reduction in the transcription and/or translation of a PQL nucleic acid. Modulating also comprises introducing expression of a PQL nucleic acid not normally found in a particular cell; or the substantially complete inhibition (eg. knockout) of expression of a PQL nucleic acid in a cell that normally has such activity.

The present invention contemplates any means by which the expression of a PQL nucleic acid may be modulated. For example, exemplary methods for modulating the expression of a PQL nucleic acid include, for example: genetic modification of the cell to upregulate or downregulate endogenous PQL nucleic acid expression; genetic modification by transformation with a PQL nucleic acid; genetic modification to increase the copy number of a PQL nucleic acid in the cell; administration of a nucleic acid molecule to the cell which modulates expression of an endogenous PQL nucleic acid in the cell; and the like.

In some embodiments, the expression of a PQL nucleic acid is modulated by genetic modification of the cell. The term “genetically modified”, as used herein, should be understood to include any genetic modification that effects an alteration in the expression of a PQL nucleic acid in the genetically modified cell relative to a non-genetically modified form of the cell. Exemplary types of genetic modification include: random mutagenesis such as transposon, chemical, UV and phage mutagenesis together with selection of mutants which overexpress or underexpress an endogenous PQL nucleic acid; transient or stable introduction of one or more nucleic acid molecules into a cell which direct the expression and/or overexpression of PQL nucleic acid in the cell; modulation of an endogenous PQ loop repeat polypeptide by site-directed mutagenesis of an endogenous PQL nucleic acid; introduction of one or more nucleic acid molecules which inhibit the expression of an endogenous PQL nucleic acid in the cell, eg. a cosuppression construct or an RNAi construct; and the like.

In some embodiments, the present invention contemplates increasing the level of a PQ loop repeat polypeptide in a cell, by introducing the expression of a PQL nucleic acid into the cell, upregulating the expression of a PQL nucleic acid in the cell and/or increasing the copy number of a PQL nucleic acid in the cell.

Methods for transformation and expression of an introduced nucleotide sequence in various cell types are well known in the art, and the present invention contemplates the use of any suitable method.

However, by way of example with regard to the transformation of plant cells, reference is made to Zhao et al. (Mol Breeding DOI 10.1007/s11032-006-9005-6, 2006), Katsuhara et al. (Plant Cell Physiol 44(12): 1378-1383, 2003), Ohta et al. (FEBS Letters 532: 279-282, 2002) and Wu et al. (Plant Science 169: 65-73, 2005).

In further embodiments the present invention also provides methods for down-regulating expression of a PQL nucleic acid in a cell. For example, with the identification of PQL nucleic acid sequences, the present invention also facilitates methods such as knockout or knockdown of a PQL nucleic acid in a cell using methods including, for example:
(i) insertional mutagenesis including knockout or knockdown of a nucleic acid in a cell by homologous recombination with a knockout construct (for an example of targeted gene disruption see Terada et al., Nat. Biotechnol. 20: 1030-1034, 2002);
(ii) post-transcriptional gene silencing (PTGS) or RNAi of a nucleic acid in a cell (for review of PTGS and RNAi see Sharp, Genes Dev. 15(5): 485-490, 2001; and Hannon, Nature 418: 244-51, 2002);
(iii) transformation of a cell with an antisense construct directed against a nucleic acid (for examples of antisense suppression see van der Krol et al., Nature 333: 866-869; van der Krol et al., BioTechniques 6: 958-967; and van der Krol et al., Gen. Genet. 220: 204-212);
(iv) transformation of a cell with a co-suppression construct directed against a nucleic acid (for an example of co-suppression see van der Krol et al., Plant Cell 2(4): 291-299);
(v) transformation of a cell with a construct encoding a double stranded RNA directed against a nucleic acid (for an example of dsRNA mediated gene silencing see Waterhouse et al., Proc. Natl. Acad. Sci. USA 95: 13959-13964, 1998);
(vi) transformation of a cell with a construct encoding an siRNA or hairpin RNA directed against a nucleic acid (for an example of siRNA or hairpin RNA mediated gene silencing see Lu et al., Nucl. Acids Res. 32(21): e171; doi:10.1093/nar/gnh170, 2004); and
(vii) insertion of a miRNA target sequence such that it is in operable connection with a nucleic acid (for an example of miRNA mediated gene silencing see Brown et al., Blood 110(13): 4144-4152, 2007).

The present invention also facilitates the downregulation of a PQL nucleic acid in a cell via the use of synthetic oligonucleotides, for example, siRNAs or microRNAs directed against a PQL nucleic acid (for examples of synthetic siRNA mediated silencing see Caplen et al., Proc. Natl. Acad. Sci. USA 98: 9742-9747, 2001; Elbashir et al., Genes Dev. 15: 188-200, 2001; Elbashir et al., Nature 411: 494-498, 2001; Elbashir et al., EMBO J. 20: 6877-6888, 2001; and Elbashir et al., Methods 26: 199-213, 2002).

In addition to the examples above, the introduced nucleic acid may also comprise a nucleotide sequence which is not directly related to a PQL nucleic acid but, nonetheless, may directly or indirectly modulate the expression of a PQL nucleic acid in a cell. Examples include nucleic acid molecules that encode transcription factors or other proteins which promote or suppress the expression of an endogenous PQL nucleic acid molecule in a cell; and other non-translated RNAs which directly or indirectly promote or suppress endogenous PQ loop repeat polypeptide expression and the like.

In order to effect expression of an introduced nucleic acid in a genetically modified cell, where appropriate, the introduced nucleic acid may be operably connected to one or more transcriptional control sequences and/or promoters, such as a native PQL nucleic acid promoter or a heterologous promoter.

The term “transcriptional control sequence” should be understood to include any nucleic acid sequence which effects the transcription of an operably connected nucleic acid. A transcriptional control sequence may include, for example, a leader, polyadenylation sequence, promoter, enhancer or upstream activating sequence, and transcription terminator. Typically, a transcriptional control sequence at least includes a promoter. The term “promoter” as used herein, describes any nucleic acid which confers, activates or enhances expression of a nucleic acid molecule in a cell.

In some embodiments, at least one transcriptional control sequence is operably connected to a PQL nucleic acid. For the purposes of the present specification, a transcriptional control sequence is regarded as “operably connected” to a given gene or other nucleotide sequence when the transcriptional control sequence is able to promote, inhibit or otherwise modulate the transcription of the gene or other nucleotide sequence.

A promoter may regulate the expression of an operably connected nucleotide sequence constitutively, or differentially, with respect to the cell, tissue, organ or developmental stage at which expression occurs, in response to external stimuli such as physiological stresses, pathogens, or metal ions, amongst others, or in response to one or more transcriptional activators. As such, the promoter used in accordance with the methods of the present invention may include, for example, a constitutive promoter, an inducible promoter, a tissue-specific promoter or an activatable promoter.

The present invention contemplates the use of any promoter which is active in a cell of interest. As such, a wide array of promoters which are active in any of bacteria, fungi, animal cells or plant cells would be readily ascertained by one of ordinary skill in the art.

However, in some embodiments, plant cells may be used. Therefore, in these embodiments, plant-active constitutive, inducible, tissue-specific or activatable promoters may be used.

Plant constitutive promoters typically direct expression in nearly all tissues of a plant and are largely independent of environmental and developmental factors. Examples of constitutive promoters that may be used in accordance with the present invention include plant viral derived promoters such as the Cauliflower Mosaic Virus 35S and 19S (CaMV 35S and CaMV 19S) promoters; bacterial plant pathogen derived promoters such as opine promoters derived from Agrobacterium spp., eg. the Agrobacterium-derived nopaline synthase (nos) promoter; and plant-derived promoters such as the rubisco small subunit gene (rbcS) promoter, the plant ubiquitin promoter (Pubi) and the rice actin promoter (Pact).

“Inducible” promoters include, but are not limited to, chemically inducible promoters and physically inducible promoters. Chemically inducible promoters include promoters which have activity that is regulated by chemical compounds such as alcohols, antibiotics, steroids, metal ions or other compounds. Examples of chemically inducible promoters include: alcohol regulated promoters (eg. see European Patent 637 339); tetracycline regulated promoters (eg. see U.S. Pat. No. 5,851,796 and U.S. Pat. No. 5,464,758); steroid responsive promoters such as glucocorticoid receptor promoters (eg. see U.S. Pat. No. 5,512,483), estrogen receptor promoters (eg. see European Patent Application 1 232 273), ecdysone receptor promoters (eg. see U.S. Pat. No. 6,379,945) and the like; metal-responsive promoters such as metallothionein promoters (eg. see U.S. Pat. No. 4,940,661, U.S. Pat. No. 4,579,821 and U.S. Pat. No. 4,601,978); and pathogenesis related promoters such as chitinase or lysozyme promoters (eg. see U.S. Pat. No. 5,654,414) or PR protein promoters (eg. see U.S. Pat. No. 5,689,044, U.S. Pat. No. 5,789,214, Australian Patent 708850, U.S. Pat. No. 6,429,362).

The inducible promoter may also be a physically regulated promoter which is regulated by non-chemical environmental factors such as temperature (both heat and cold), light and the like. Examples of physically regulated promoters include heat shock promoters (eg. see U.S. Pat. No. 5,447,858, Australian Patent 732872, Canadian Patent Application 1324097); cold inducible promoters (eg. see U.S. Pat. No. 6,479,260, U.S. Pat. No. 6,184,443 and U.S. Pat. No. 5,847,102); light inducible promoters (eg. see U.S. Pat. No. 5,750,385 and Canadian Patent 132 1563); light repressible promoters (eg. see New Zealand Patent 508103 and U.S. Pat. No. 5,639,952).

“Tissue specific promoters” include promoters which are preferentially or specifically expressed in one or more specific cells, tissues or organs in an organism and/or one or more developmental stages of the organism. It should be understood that a tissue specific promoter also be constitutive or inducible.

Examples of plant tissue specific promoters include: root specific promoters such as those described in US Patent Application 2001047525; fruit specific promoters including ovary specific and receptacle tissue specific promoters such as those described in European Patent 316 441, U.S. Pat. No. 5,753,475 and European Patent Application 973 922; and seed specific promoters such as those described in Australian Patent 612326 and European Patent application 0 781 849 and Australian Patent 746032.

The promoter may also be a promoter that is activatable by one or more transcriptional activators, referred to herein as an “activatable promoter”. For example, the activatable promoter may comprise a minimal promoter operably connected to an Upstream Activating Sequence (UAS), which comprises, inter glia, a DNA binding site for one or more transcriptional activators.

As referred to herein the term “minimal promoter” should be understood to include any promoter that incorporates at least an RNA polymerase binding site and, optionally a TATA box and transcription initiation site and/or one or more CAAT boxes. In some embodiments wherein the cell is a plant cell, the minimal promoter may be derived from the Cauliflower Mosaic Virus 35S (CaMV 35S) promoter. The CaMV 35S derived minimal promoter may comprise, for example, a sequence that substantially corresponds to positions −90 to +1 (the transcription initiation site) of the CaMV 35S promoter (also referred to as a −90 CaMV 35S minimal promoter), −60 to +1 of the CaMV 35S promoter (also referred to as a −60 CaMV 35S minimal promoter) or −45 to +1 of the CaMV 35S promoter (also referred to as a −45 CaMV 35S minimal promoter).

As set out above, the activatable promoter may comprise a minimal promoter fused to an Upstream Activating Sequence (UAS). The UAS may be any sequence that can bind a transcriptional activator to activate the minimal promoter. Exemplary transcriptional activators include, for example: yeast derived transcription activators such as Gal4, Pdr1, Gcn4 and Ace1; the viral derived transcription activator, VP16; Hap1 (Hach et al., J Biol Chem 278: 248-254, 2000); Gaf1 (Hoe et al., Gene 215(2): 319-328, 1998); E2F (Albani et al., J Biol Chem 275: 19258-19267, 2000); HAND2 (Dai and Cserjesi, J Biol Chem 277: 12604-12612, 2002); NRF-1 and EWG (Herzig et al., J Cell Sci 113: 4263-4273, 2000); P/CAF (Itoh et al., Nucl Acids Res 28: 4291-4298, 2000); MafA (Kataoka et al., J Biol Chem 277: 49903-49910, 2002); human activating transcription factor 4 (Liang and Hai, J Biol Chem 272: 24088-24095, 1997); Bcl10 (Liu et al., Biochem Biophys Res Comm 320(1): 1-6, 2004); CREB-H (Omori et al., Nucl Acids Res 29: 2154-2162, 2001); ARR1 and ARR2 (Sakai et al., Plant J 24(6): 703-711, 2000); Fos (Szuts and Bienz, Proc Natl Acad Sci USA 97: 5351-5356, 2000); HSF4 (Tanabe et al., J Biol Chem 274: 27845-27856, 1999); MAML1 (Wu et al., Nat Genet. 26: 484-489, 2000).

In some embodiments, the UAS comprises a nucleotide sequence that is able to bind to at least the DNA-binding domain of the GAL4 transcriptional activator.

The transcriptional control sequence may also include a terminator. The term “terminator” refers to a DNA sequence at the end of a transcriptional unit which signals termination of transcription. Terminators are 3′-non-translated DNA sequences generally containing a polyadenylation signal, which facilitate the addition of polyadenylate sequences to the 3′-end of a primary transcript. As with promoter sequences, the terminator may be any terminator sequence which is operable in the cells, tissues or organs in which it is intended to be used. Examples of suitable terminator sequences which may be useful in plant cells include: the nopaline synthase (nos) terminator, the CaMV 35S terminator, the octopine synthase (ocs) terminator, potato proteinase inhibitor gene (pin) terminators, such as the pinII and pinIII terminators and the like.

In a second aspect, the present invention provides a cell with a modulated rate, level or pattern of cation flux across a membrane of the cell, wherein said modulation is the result of modulated expression of a PQ-loop repeat polypeptide in the cell.

The term “cell”, as used herein, should be understood to include any cell type, including bacteria, archaea and eukaryotic cells including, for example, animal, plant and fungal cells. In some embodiments, the cell may include, for example, a plant cell, a monocot plant cell, or a cereal crop plant cell.

In some embodiments, the cell of the second aspect of the invention is produced according to a method of the first aspect of the invention. In further embodiments, the cell of the second aspect of the invention is genetically modified as described above with reference to the first aspect of the invention.

As referred to herein, a “genetically modified cell” comprises a cell that is genetically modified with respect to the wild type of the cell. As such, a genetically modified cell may be a cell which has itself been genetically modified and/or the progeny of such a cell which retains a modification with respect to the wild type of the cell.

In a third aspect, the present invention provides a multicellular structure comprising one or more cells of the second aspect of the invention.

As referred to herein, a “multicellular structure” includes any aggregation of one or more cells. As such, a multicellular structure specifically encompasses tissues, organs, whole organisms and parts thereof. Furthermore, a multicellular structure should also be understood to encompass multicellular aggregations of cultured cells such as colonies, plant calli, liquid or suspension cultures and the like.

As mentioned above, in some embodiments, the cell is a plant cell and, as such, the present invention includes a whole plant, plant tissue, plant organ, plant part, plant reproductive material or cultured plant tissue (eg. callus or suspension culture), comprising one or more plant cells according to the third aspect of the invention. In further embodiments, the cell is a monocot cell or a cereal crop cell and, thus, the present invention also specifically includes a whole plant, plant tissue, plant organ, plant part, plant reproductive material or cultured plant tissue (eg. callus or suspension culture), comprising one or more monocot or cereal crop plant cells.

In a fourth aspect, the present invention provides a nucleic acid construct or vector comprising a PQL nucleic acid as hereinbefore described.

The nucleic acid construct or vector of the present invention may be composed of any polyribonucleotide or polydeoxyribonucleotide, which may be unmodified RNA or DNA or modified RNA or DNA. For example, the construct or vector can be composed of single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, the construct or vector may comprise triple-stranded regions comprising RNA or DNA or both RNA and DNA. The construct or vector may also contain one or more modified bases or DNA or RNA backbones modified for stability or for other reasons. “Modified” bases include, for example, tritylated (or other labelled) bases and unusual bases such as inosine. A variety of modifications can be made to DNA and RNA; thus, “nucleic acid” embraces chemically, enzymatically, or metabolically modified forms.

In some embodiments, the construct is an expression construct which may be used to effect expression of a PQ loop repeat polypeptide in a cell as described with reference to the first aspect of the invention.

Thus, the vector or construct may further comprise one or more of: an origin of replication for one or more hosts; a selectable marker gene which is active in one or more hosts; and/or one or more transcriptional control sequences.

As used herein, the term “selectable marker gene” includes any gene that confers a phenotype on a cell in which it is expressed, to facilitate the identification and/or selection of cells which are transfected or transformed with a genetic construct.

A range of nucleotide sequences encoding suitable selectable markers are known in the art. Exemplary nucleotide sequences that encode selectable markers include, for example: antibiotic resistance genes such as ampicillin-resistance genes, tetracycline-resistance genes, kanamycin-resistance genes, the AURI-C gene which confers resistance to the antibiotic aureobasidin A, neomycin phosphotransferase genes (eg. nptI and nptII) and hygromycin phosphotransferase genes (eg. hpt); herbicide resistance genes including glufosinate, phosphinothricin or bialaphos resistance genes such as phosphinothricin acetyl transferase encoding genes (eg. bar), glyphosate resistance genes including 3-enoyl pyruvyl shikimate 5-phosphate synthase encoding genes (eg. aroA), bromyxnil resistance genes including bromyxnil nitrilase encoding genes, sulfonamide resistance genes including dihydropterate synthase encoding genes (eg. sul) and sulfonylurea resistance genes including acetolactate synthase encoding genes; enzyme-encoding reporter genes such as GUS and chloramphenicolacetyltransferase (CAT) encoding genes; fluorescent reporter genes such as the green fluorescent protein-encoding gene; and luminescence-based reporter genes such as the luciferase gene, amongst others.

Furthermore, it should be noted that the selectable marker gene may be a distinct open reading frame in the construct or may be expressed as a fusion protein with another polypeptide (eg. a PQ loop repeat polypeptide).

As set out above, the nucleic acid construct or vector may also comprise one or more transcriptional control sequences as described above. In some embodiments, at least one transcriptional control sequence is operably connected to the nucleic acid sequence of the first aspect of the invention as hereinbefore described.

As mentioned above, the control sequences may also include a terminator as hereinbefore described.

The construct may further include nucleotide sequences intended for the maintenance and/or replication of the genetic construct in prokaryotes or eukaryotes and/or the integration of the genetic construct or a part thereof into the genome of a eukaryotic or prokaryotic cell.

In some embodiments, the vector or construct is adapted to be at least partially transferred into a plant cell via Agrobacterium-mediated transformation. Accordingly, in some embodiments, the construct may comprise left and/or right T-DNA border sequences.

Suitable T-DNA border sequences would be readily ascertained by one of skill in the art. However, the term “T-DNA border sequences” should be understood to include, for example, any substantially homologous and substantially directly repeated nucleotide sequences that delimit a nucleic acid molecule that is transferred from an Agrobacterium sp. cell into a plant cell susceptible to Agrobacterium-mediated transformation. By way of example, reference is made to the paper of Peralta and Ream (Proc. Natl. Acad. Sci. USA, 82(15): 5112-5116, 1985) and the review of Gelvin (Microbiology and Molecular Biology Reviews, 67(1): 16-37, 2003).

In some embodiments, the vector or construct is adapted to be transferred into a plant via Agrobacterium-mediated transformation. However, the present invention also contemplates any suitable modifications to the genetic construct that facilitate bacterial mediated insertion into a plant cell via bacteria other than Agrobacterium sp., for example as described in Broothaerts et al. (Nature 433: 629-633, 2005).

Those skilled in the art will be aware of how to produce the constructs described herein and of the requirements for obtaining their expression in a specific cell or cell-type. The genetic manipulations required to perform the present invention may require the propagation of a genetic construct described herein or a derivative thereof in a prokaryotic cell such as an E. coli cell or a plant cell or an animal cell.

In a fifth aspect, the present invention provides a genetically modified cell comprising a nucleic acid construct or vector of the fourth aspect of the invention.

The nucleic acid may be introduced using any method known in the art which is suitable for the cell type being used.

In embodiments where the cell is a plant cell, suitable methods for introduction of a nucleic acid molecule may include, for example: Agrobacterium-mediated transformation, other bacterially-mediated transformation (see Broothaerts et al., 2005, supra) microprojectile bombardment based transformation methods and direct DNA uptake based methods. Roa-Rodriguez et al. (Agrobacterium-mediated transformation of plants, 3rd Ed. CAMBIA Intellectual Property Resource, Can berra, Australia, 2003) review a wide array of suitable Agrobacterium-mediated plant transformation methods for a wide range of plant species. Microprojectile bombardment may also be used to transform plant tissue and methods for the transformation of plants, particularly cereal plants, and such methods are reviewed by Casas et al. (Plant Breeding Rev. 13: 235-264, 1995). Direct DNA uptake transformation protocols such as protoplast transformation and electroporation are described in detail in Galbraith et al. (eds.), Methods in Cell Biology Vol. 50, Academic Press, San Diego, 1995). In addition to the methods mentioned above, a range of other transformation protocols may also be used. These include infiltration, electroporation of cells and tissues, electroporation of embryos, microinjection, pollen-tube pathway, silicon carbide- and liposome mediated transformation. Methods such as these are reviewed by Rakoczy-Trojanowska (Cell. Mol. Biol. Lett. 7: 849-858, 2002). A range of other plant transformation methods may also be evident to those of skill in the art.

The construct or vector referred to above may be maintained in the cell as a DNA molecule, as part of an episome (eg. a plasmid, cosmid, artificial chromosome or the like) or it may be integrated into the genomic DNA of a cell.

As used herein, the term “genomic DNA” should be understood in its broadest context to include any and all DNA that makes up the genetic complement of a cell. As such, the genomic DNA of a cell should be understood to include chromosomes, mitochondrial DNA, plastid DNA, chloroplast DNA, endogenous plasmid DNA and the like. As such, the term “genomically integrated” contemplates chromosomal integration, mitochondrial DNA integration, plastid DNA integration, chloroplast DNA integration, endogenous plasmid integration, and the like.

The term “cell”, as used herein, should be understood to include any cell type, including bacteria, archaea and eukaryotic cells including, for example, animal, plant and fungal cells. In some embodiments, the cell may include, for example, a plant cell, a monocot plant cell, or a cereal crop plant cell.

Furthermore, in a sixth aspect, the present invention provides a multicellular structure comprising one or more cells of the fifth aspect of the invention.

As mentioned above, in some embodiments, the cell is a plant cell and, as such, the present invention includes a whole plant, plant tissue, plant organ, plant part, plant reproductive material or cultured plant tissue (eg. callus or suspension culture), comprising one or more plant cells according to the fifth aspect of the invention. In further embodiments, the cell is a monocot cell or a cereal crop cell and, thus, the present invention also specifically contemplates a whole plant, plant tissue, plant organ, plant part, plant reproductive material or cultured plant tissue (eg. callus or suspension culture), comprising one or more monocot or cereal crop plant cells.

In a seventh aspect, the present invention provides a method for ascertaining the cation sensitivity or tolerance of an organism, the method comprising determining the expression of a PQ loop repeat polypeptide in one or more cells of the organism.

In some embodiments, a relatively high level of expression is associated with cation sensitivity in the organism.

In some embodiments, a relatively high level of expression in all cells of the organism is associated with cation tolerance in the organism.

In another embodiment, a relatively low level of expression is associated with cation tolerance in the organism.

As referred to herein, “determining the expression of a PQ loop repeat polypeptide” includes determining the level and/or activity of the PQ loop repeat polypeptide itself, and/or the level, activity, transcription or translation of a PQL nucleic acid.

Methods for determining the level and/or pattern of expression of a nucleic acid or polypeptide are known in the art. Exemplary methods of the detection of RNA expression include methods such as quantitative or semi-quantitative reverse-transcriptase PCR (eg. see Burton et al., Plant Physiology 134: 224-236, 2004), in-situ hybridization (eg. see Linnestad et al., Plant Physiology 118: 1169-1180, 1998); northern blotting (eg. see Mizuno et al., Plant Physiology 132: 1989-1997, 2003); and the like. Exemplary methods for determining the expression of a polypeptide include Western blotting (eg. see Fido et al., Methods Mol. Biol. 49: 423-37, 1995); ELISA (eg. see Gendloff et al., Plant Molecular Biology 14: 575-583); immunomicroscopy (eg. see Asghar et al., Protoplasma 177: 87-94, 1994) and the like. In another embodiment, the expression of a PQL nucleic acid sequence may be determined by determining the number of PQL nucleic acids present in the genomic DNA of one or more cells of the organism.

In some embodiments, the method of the seventh aspect of the invention is adapted to ascertaining the cation sensitivity or tolerance of a plant. In further embodiments, the method of the seventh aspect of the invention is adapted to ascertaining the cation sensitivity or tolerance of, for example, a monocot plant or a cereal crop plant.

In further embodiments, the method of the seventh aspect of the invention may be used to ascertain the cation sensitivity or tolerance of an organism and then select individual organisms on the basis of the ascertained level of cation sensitivity or tolerance. For example, in the case of plants, plants having increased cation tolerance may be selected for planting in high cation soils or may be selected for breeding programs to produce cation tolerant cultivars of the plant.

Finally, reference is made to standard textbooks of molecular biology that contain methods for carrying out basic techniques encompassed by the present invention, including DNA restriction and ligation for the generation of the various genetic constructs described herein. See, for example, Maniatis et al., Molecular Cloning: A Laboratory Manual (Cold Spring Harbor Laboratory Press, New York, 1982) and Sambrook et al. (2000, supra).

The present invention is further described by the following non-limiting examples:

### BRIEF DESCRIPTION OF THE FIGURES

FIG. 1 shows ¹⁴C labeled MA flux in S. cerevisiae strain 31019b over expressing YDR352w, YOL092w or the pYES3 control. S. cerevisiae strain 31019b (mep1Δ, mep2Δ, mep3Δ, ura3Δ) (Marini et al., EMBO J. 13: 3456-3463, 2000) was transformed with pYES3-DEST (Smith et al., Proceedings of the National Academy of Sciences 92: 9373-9377, 1995, modified by M. Shelden (unpublished)) containing either YDR352w, YOL092w or no insert. Cells were grown and resuspended in a 20 mM KPO₄⁻ buffer at pH 7.0. 50 mM ¹⁴C labeled MA was added at T=0 and cells sampled by filtration through a 0.45 μm membrane. MA content was determined relative to total cell protein. Data significance was determined through two-way ANOVA and significance shown is relative to the empty vector control. YDR352w and YOL092w differ significantly only after 30 minutes of flux. (n=5). (*=P value<0.005; **=P value<0.001; ***=P value<0.0001).

FIG. 2 shows concentration dependant ¹⁴C labelled MA flux. S. cerevisiae strain 31019b (mep1Δ, mep2Δ, mep3Δ, ura3Δ) (Marini et al., 2000, supra) was transformed with pYES3 (Smith et al., 1995, supra) containing either YDR352w, YOL092w or no insert. Cells were grown and resuspended in a 20 mM KPO₄⁻ buffer at pH 7.0. ¹⁴C labelled MA was added at various concentrations and cells sampled by filtration through a 0.45 μm membrane. MA content was determined relative to total cell protein. Data significance was determined through two-way ANOVA and significance shown is relative to the empty vector control. (n=6).

FIG. 3 shows time dependant flux of ²²Na Flux data into Saccharomyces cerevisiae strain 31019b. S. cerevisiae strain 31019b (mep1,2,3Δ, ura3Δ) (Marini et al., 2000, supra) was transformed with pYES3 (Smith et al., 1995, supra) containing either YDR352w, YOL092w or no insert. Panel A shows overall data whereas panel B shows Na⁺ accumulation above that of the empty vector control, calculated by subtracting empty vector values from those of yeast expressing PQ loop repeat proteins. Cells were grown and resuspended in a 20 mM KPO₄⁻ buffer at pH 7.0. 50 mM ²²Na labelled NaCl was added at T=0 and cells sampled by filtration through a 0.45 μm membrane. MA content was determined relative to total cell protein. Data significance was determined through two way ANOVA and significance shown is relative to the empty vector control. Greater variation most likely exists in the data as a result of native Na⁺ conducting proteins.

FIG. 4 shows optimisation of bath solutions to analyse cation flux in Xenopus laevis oocytes expressing YDR352w. Xenopus laevis oocytes were injected with either YDR352w cRNA or nuclease free H₂O. Oocytes were exposed to the standard voltage protocol in various bath solutions. A) 100 mM Choline Cl, 2 mM MgCl₂, 1 mM CaCl₂, 5 mM MES/Tris pH 6.5; B) 200 mM Mannitol, 2 mM MgCl₂, 1 mM CaCl₂, pH 7.0 Tris; C) 200 mM Mannitol, 2 mM MgCl₂, 2 mM BaCl₂, pH 7.0 Tris; D) 200 mM Mannitol, 5 mM MES/Tris pH 7.0. Buffer D) showed no evidence of the Ca²⁺ activated Cl channel and was used as a base for further experiments (n=minimum of 4).

FIGS. 5 A to C shows the influence of external Na⁺ concentration on current conductance of oocytes expressing S. cerevisiae PQ loop repeat proteins. Oocytes injected with cRNA from YDR352w or YOL092w were compared to oocytes injected with nuclease free H₂O over a range of Na⁺ concentrations. Concentrations were 100 mM NaCl (A), 10 mM NaCl (B) and 1 mM NaCl (C) each in standard buffer as described in example 7. As concentrations of Na increase, Eᵣₑᵥ shifts towards theoretical E_{Na} as opposed to E_{Cl}, confirming that Na⁺ is the fluxed ion. (n=5).

FIGS. 5 D and E shows a comparison of induced currents in Xenopus laevis oocytes expressing S. cerevisiae PQ loop repeat proteins as a function of external Na⁺ concentration. I/V relationship between oocytes expressing YDR352w (A) or YOL092w (B) bathed in buffers containing different Na⁺ concentrations. Buffers are mannitol/MES based with NaCl added as required. As the external Na⁺ concentration increases the Eᵣₑᵥ shifts to the right, towards the calculated E_{Na} value. This confirms Na⁺ is the carried ion as opposed to Cl⁻, which would cause E_{Rev} to shift to the left as concentrations increased. (n=5).

FIG. 6 shows the effect of TEA⁺ on Na⁺ flux through YDR352w and YOL092w. The influence of the K⁺ channel blocker TEA⁺ on YDR352w and YOL092w was investigated. The presence of TEA (Buffer (A): 10 mM TEA-OH, 90 mM NaCl, 5 mM MES/Tris pH 7.0) showed greater current compared to those oocytes in a 90 mM NaCl alone (Buffer (B): 90 mM NaCl, 5 mM MES/Tris). This may be due to flux of TEA⁺ through these proteins or the result of TEA⁺ acting as an agonist to native NSCCs.

FIG. 7A shows NSCC activity for Xenopus laevis oocytes expressing YDR352w or YOL092w. I/V relationship of YOL092w (A) or YDR352w (B) expressed in oocytes, bathed in various cation buffers. Bath solution was a base of 200 mM Mannitol and 5 mM MES/Tris at pH 7.0 with cations added as above.

FIG. 7B shows NSCC activity with comparison to the H₂O injected control. Comparison of cation currents through oocytes injected with YDR352w or YOL092w cRNA or H₂O injected controls. Data for bathing solutions containing 100 mM Na⁺ (A), 100 mM NH₄⁺ (C) and 100 mM Choline (D) are significantly different to the water injected control. Data for oocytes bathed in 100 mM K⁺ (B) is not significantly different to the water injected control as a result of a large amount of native Xenopus oocyte activity (n=5 for (A), (B), and (D). For (C), n=2).

FIG. 8 shows that changing counter anion from Cl⁻ to SO₄²⁻ influences current traces. The substitution of 100 mM NaCl bath solution (Buffer (A): 100 mM NaCl, 5 mM MES/Tris pH 7.0) with a 50 mM Na₂SO₄ bath solution (Buffer (B): 50 mM Na₂SO₄, 5 mM MES/Tris pH 7.0) slightly influenced Na⁺ flux through YOL092w but did not affect flux catalysed by expression of YDR352w. The inhibition of YOL092w flux in (B) may be a result of altered SO₄²⁻ flux across membranes. (n=5).

FIG. 9 panels A, B and C show the influence of differing external Ca²⁺ concentration on Na⁺ conductance through YDR352w or YOL0921w, expressed in Xenopus oocytes. Induced current as a result of Na⁺ flux was reduced by the presence of Ca²⁺ in the bath solution. Current at 0 mM Ca²⁺ (I/V curve (A): 100 mM NaCl, 5 mM MES/Tris pH 7.0) was reduced by approximately 50% upon replacement of the bath solution with a 2 mM Ca²⁺ buffer (I/V curve (B): 100 mM NaCl, 2 mM CaCl₂, 5 mM MES/Tris pH 7.0). Exchanging the bath solution to a 10 mM Ca²⁺ buffer (I/V curve (C): 100 mM NaCl, 10 mM CaCl₂, 5 mM MES/Tris) reduces current by approximately another 20%. Overall, 60% of the initial 0 mM Ca²⁺ flux is reduced with the addition of 10 mM CaCl₂. In all graphs the I/V curve for PQ loop expressing oocytes is significantly different from the H₂O injected control. n=5.

FIG. 9 panels D, E and F show the effect of Ca²⁺ concentration on Na⁺ flux through YDR352w and YOL092w expressed in Xenopus oocytes. Na⁺ flux through Xenopus oocytes expressing either YDR352w (A) or YOL092w (B) was measured by voltage clamping in 100 mM NaCl bathing solution with various Ca²⁺ concentrations. Na⁺ flux was greatest in the absence of Ca²⁺ and was progressively decreased with the addition of buffers containing 2 mM CaCl₂ or 10 mM CaCl₂. Reversal potential shifts towards positive potentials with increased Ca²⁺ concentrations together, towards theoretical E_{Ca} together with previous induction of the Ca²⁺ activated Cl⁻ channel suggest the Ca²⁺ inhibition of Na⁺ flux may be a function of Ca²⁺ being transported. (n=5)

FIG. 10 shows representative traces for the data shown in FIG. 4. Representative traces of current recorded in oocytes injected with YDR352w cRNA (A, B, C and D) or nuclease free H₂O (E, F, G, and H). Traces correspond with the I/V curves of the previous figure with A and E for FIG. 4 buffer A, B and F for buffer B, C and G for buffer C and D and H for Buffer D.

FIG. 11A shows MA⁺ toxicity and uptake in S. cerevisiae strain 31019b transformed with the empty pYES3 vector. S. cerevisiae strain 31019b was transformed with empty pYES3-DEST vector and cells were grown and plated. Grensons minimal media (Grenson, Biochimica et Biophysica Acta 127: 339-346, 1966) was supplemented with 0.1% L-proline, 100 mM MA and 2% galactose with Ca²⁺ and pH modulated as follows—1: 10 mM Ca²⁺ pH 6.5; 2) 0.2 mM Ca²⁺ pH 6.5; 3) 10 mM Ca²⁺ pH 7.0, 4) 0.2 mM Ca²⁺ pH 7.0; 5) YNB+2% glucose as a loading control.

FIG. 11B shows MA⁺ toxicity and uptake in S. cerevisiae strain 31019b transformed with the YDR352w in the galactose inducible vector BG1805. S. cerevisiae strain 31019b was transformed with the yeast expression vector BG1805 containing YDR352w. (A) Cells were grown and plated. Grensons minimal media (Grenson, 1966, supra) was supplemented with 0.1% L-proline, 100 mM MA and 2% galactose with Ca²⁺ and pH modulated as follows—1: 10 mM Ca²⁺ pH 6.5; 2) 0.2 mM Ca²⁺ pH 6.5; 3) 10 mM Ca²⁺ pH 7.0, 4) 0.2 mM Ca²⁺ pH 7.0; 5) YNB+2% glucose as a loading control. (B) Transformed 31019b cells were incubated in reaction buffer containing 0.5 mM ¹⁴C-MA, 20 mM KPO₄⁻ and 2% D-galactose at pH 7.0. Net uptake of ¹⁴C MA was measured in harvested cells using a scintillation counter after 20 minutes incubation. Data presented is mean±SE (n=10).

FIG. 11C shows MA⁺ toxicity and uptake in S. cerevisiae strain 31019b transformed with YOL092w in the galactose inducible vector BG1805. S. cerevisiae strain 31019b was transformed with the yeast expression vector BG1805 containing YOL092w. (A) Cells were grown and plated. Grensons minimal media (Grenson, 1966, supra) was supplemented with 0.1% L-proline, 100 mM MA and 2% galactose with Ca²⁺ and pH modulated as follows—1: 10 mM Ca²⁺ pH 6.5; 2) 0.2 mM Ca²⁺ pH 6.5; 3) 10 mM Ca²⁺ pH 7.0, 4) 0.2 mM Ca²⁺ pH 7.0; 5) YNB+2% glucose as a loading control. (B) Transformed 31019b cells were incubated in reaction buffer containing 0.5 mM ¹⁴C-MA, 20 mM KPO₄⁻ and 2% D-galactose at pH 7.0. Net uptake of ¹⁴C MA was measured in harvested cells using a scintillation counter after 20 minutes incubation. Data presented is mean±SE (n=10).

FIG. 12 shows the voltage protocol for electrophysiological data described here.

FIG. 13 shows a CLUSTAL W alignment of S. cerevisiae YDR352w, YOL092w and YBR147w with PQ loop repeats and putative regions of interest marked. Amino acid sequences of YDR352w, YOL092w and the very similar YBR147w were aligned using the CLUSTAL-W algorithm. There is a high degree of sequence conservation, particularly in the transmembrane domains and ‘PQ loop’ regions. Putative G-protein binding regions are predicted from Chung et al. (J. Biol. Chem. 276: 40190-40201, 2001). Transmembrane domains predicted using adapted Gene3D (Buchan et al., Genome Res. 12: 503-514, 2002) in the SGD.

FIG. 14 shows a phylogenetic tree of Arabidopsis thaliana, Saccharomyces cerevisiae, Oryza sativa, Triticum aestivum, Schizosaccharomyces pombe and Homo sapiens proteins that show sequence similarity to YDR352w. Protein sequence of YDR352w was compared to protein sequence databases through the BLAST algorithm using standard parameters for NCBI. Additional protein sequences of annotated PQ loop repeat containing proteins were also retrieved. Protein sequences were aligned using the CLUSTALW algorithm and a phylogeny tree was created (MacVector, USA).

FIG. 15 shows representative time dependant current profiles of oocytes injected with YDR352w cRNA (A, B, C), YOL092w cRNA (D, E, F) or H₂O (G, H, I). Oocytes were bathed in base buffer (see methods of this chapter) with 1 mM NaCl (A, D, G), 10 mM NaCl (B, E, H) or 100 mM NaCl (C, F, I) added.

FIG. 16 shows representative time dependant current profiles of oocytes injected with YDR352w cRNA (A, B), YOL092w cRNA (C, D) or H₂O (E, F). Oocytes were bathed in base buffer (see methods of this chapter) with 100 mM NaCl (A, C, E) or 50 mM Na₂SO₄ (B, D, F).

FIG. 17 shows a phylogenetic tree of proteins that show some degree of similarity to YDR352w and YOL092w when compared to publicly available genomic databases.

FIG. 18 shows a cladogram of protein sequences of two yeast (Yol092wp and Ydr352wp), six Arabidopsis (AtPQL1-6), three rice (Os01g16170, Os07g29610 and Os12g18110), six Physcomitrella patens (Pp174957, Pp182799, Pp217317, Pp210671, Pp159185 and Pp160065) and two bacterial (FtPQL and UpPQL) PQ loop proteins (to anchor the tree). Higher plant PQ loop proteins are separated into clades I, II and III.

FIG. 19 is a diagram showing the predicted protein topology of PQ loop proteins from yeast—Yol092wp (top left), and Arabidopsis—AtPQL1, 2 and 3 (top right, bottom left, bottom right, respectively).

FIG. 20 illustrates the results of a salt sensitivity assay of Saccharomyces cerevisiae transformed with AtPQL1, AtPQL2, or AtPQL3. Tenfold serial dilutions were spotted onto SD-uracil medium supplemented with 500 mM NaCl and/or 10 mM CaCl₂. Plates were incubated at 30° C. for 2 days. Yeast expressing AtPQL1 show reduced growth rate compared to control, which is recovered by the addition of 10 mM CaCl₂. Yeast expressing AtPQL1 also showed increased salt sensitivity compared to control. Again this sensitivity could be recovered 10 mM CaCl₂, indicating that Ca²⁺ may be interacting with AtPQL1.

FIG. 21 is a graphical representation showing the growth of S. cerevisiae transformed with AtPQL1, AtPQL2, or AtPQL3. Cells were grown in SD-uracil medium supplemented with either 0 mM CaCl or 10 mM CaCl. Growth is expressed as percent over the maximum growth of control (n=3). Yeast expressing AtPQL1 show reduced growth rate compared to control, which is recovered by the addition of 10 mM CaCl₂.

FIG. 22 is a graphical representation showing the reduction of growth of S. cerevisiae transformed with AtPQL1, AtPQL2, or AtPQL3. Cells were grown in SD-uracil medium supplemented with 500 mM NaCl, and either 0 mM CaCl or 10 mM CaCl. Growth is expressed as percent over the maximum growth of untreated transformants (n=3). Yeast expressing AtPQL1 show increased salt sensitivity compared to control. This sensitivity could be recovered 10 mM CaCl₂.

FIG. 23A is a graphical representation showing biomass accumulation of hydroponically grown plants with no NaCl application. K.O=gene knockout lines, amiRNA=gene knockdown lines, 35S=overexpressing lines. Transgenic lines are segregating and so contain nulls. Results are the mean±standard error of the mean.

FIG. 23B is a graphical representation showing biomass accumulation of hydroponically grown plants, after 3 days of 50 mM NaCl application. K.O=gene knockout lines, amiRNA=gene knockdown lines, 35S=overexpressing lines. Transgenic lines are segregating and so contain nulls. Results are the mean±standard error of the mean.

FIG. 24 is a graphical representation showing salinity tolerance of hydroponically grown plants. % salinity tolerance was calculated by dividing the average biomass of each line under salt stress by the average biomass of the same line under control conditions. K.O=gene knockout lines, amiRNA=gene knockdown lines, 35S=overexpressing lines. Transgenic lines are segregating and so contain nulls.

FIG. 25 shows the sodium concentrations in segregating T₂ 35S::OsPQL1 rice plants that were grown in hydroponics for 2 weeks before the addition of 75 mM NaCl for 12 days. Flame photometry of 3^{rd} fully expanded leaf determined that plants from both lines have significantly lower shoot Na⁺ than wild type (WT) Nipponbare plants (n=12). 35S-OsPQL-A and 35S-OsPQL-B are independent transformants of rice calli transformed with the same 35S::OsPQL construct.

FIG. 26 shows the localisation of AtPQL1 in tobacco epidermal cells. DNA constructs with the green fluorescent protein (GFP) fused to the AtPQL1 protein were kindly provided by Dr Anna Antmann (University of Glasgow) and transformed into tobacco epidermal cells. GFP localisation was visualised using confocal microscopy.

### Example 1

### PQ Loop Repeat Proteins as Putative viNSCCs in Saccharomyces cerevisiae

A Saccharomyces cerevisiae based screen was developed to identify putative viNSCCs. This screen was dependent on the flux of the NH₄⁺ analogue methylammonium (MA) through the viNSCC to produce a toxicity phenotype in yeast. By altering Ca²⁺ concentration and the pH of the growth media we were able to select for proteins displaying phenotypes expected of overexpressed viNSCCs.

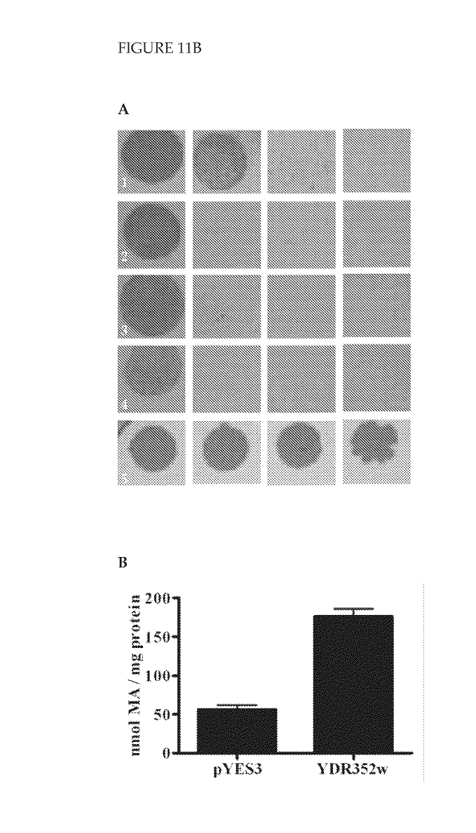

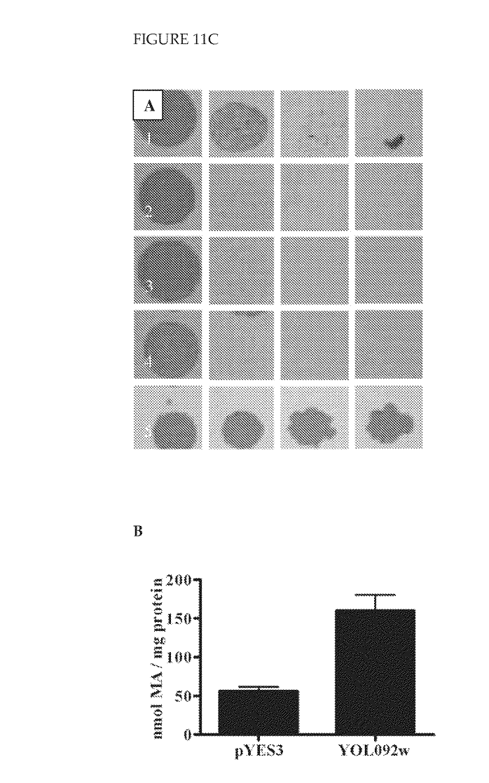

This screen identified two proteins of the PQ loop repeat class that showed the phenotypic response anticipated of overexpressing viNSCCs in yeast (FIG. 11A, B and C). A series of experiments were developed to confirm these proteins behave as viNSCCs.

### Example 2

### Cation Flux into Saccharomyces cerevisiae Over Expressing YDR352w or YOL092w

YDR352w and YOL092w were selected based upon their ability to impart a toxicity phenotype in cells of the S. cerevisiae strain 31019b in the presence of toxic MA concentrations. This strain has no functional expression of all three of its native high affinity NH₄⁺/MA transporters allowing it to survive on media containing high MA concentrations.

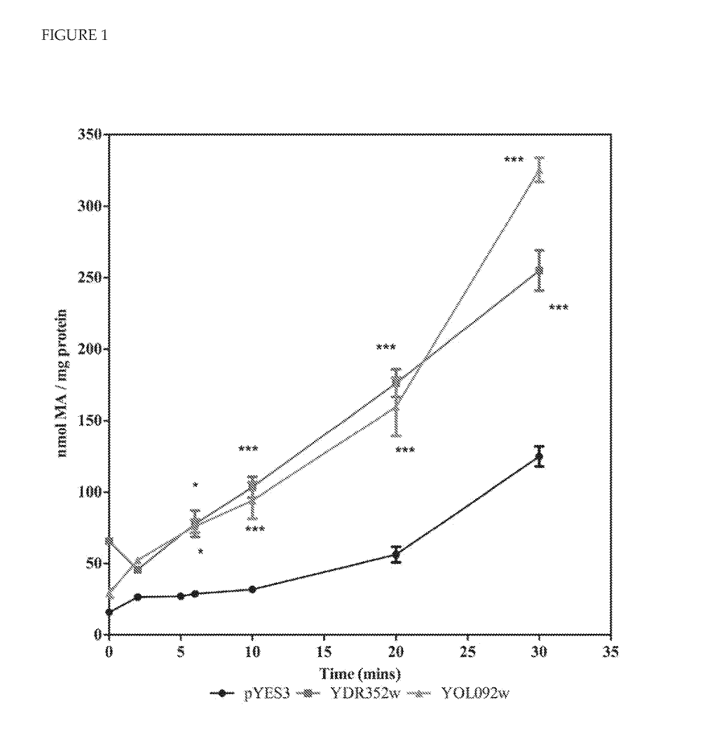

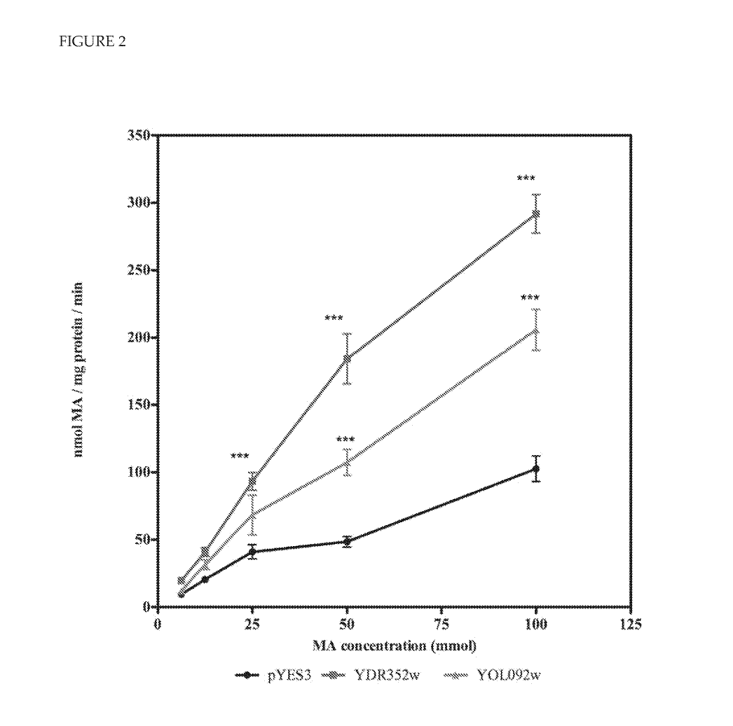

Over expression of YDR352w and YOL092w resulted in a toxicity phenotype that was relieved with increased Ca²⁺ (FIG. 11 A, B and C). To confirm this phenotype was the result of increased MA influx, ¹⁴C labelled MA flux at an external concentration of 0.5 mM MA was measured. Cells over expressing either YDR352w or YOL092w resulted in increased MA accumulation over time when compared to the empty vector control. Uptake followed an arithmetic regression and had an accumulation rate of 2.5-3 times that of the empty vector control at 30 minutes (FIG. 1). Concentration dependant MA flux through these proteins was also examined (FIG. 2). MA uptake followed an unsaturable profile significantly greater than that of the empty vector control. Cells over expressing YDR352w show a greater MA flux capacity than those over expressing YOL092w, although they both show significant LATS activity relative to the empty vector control.

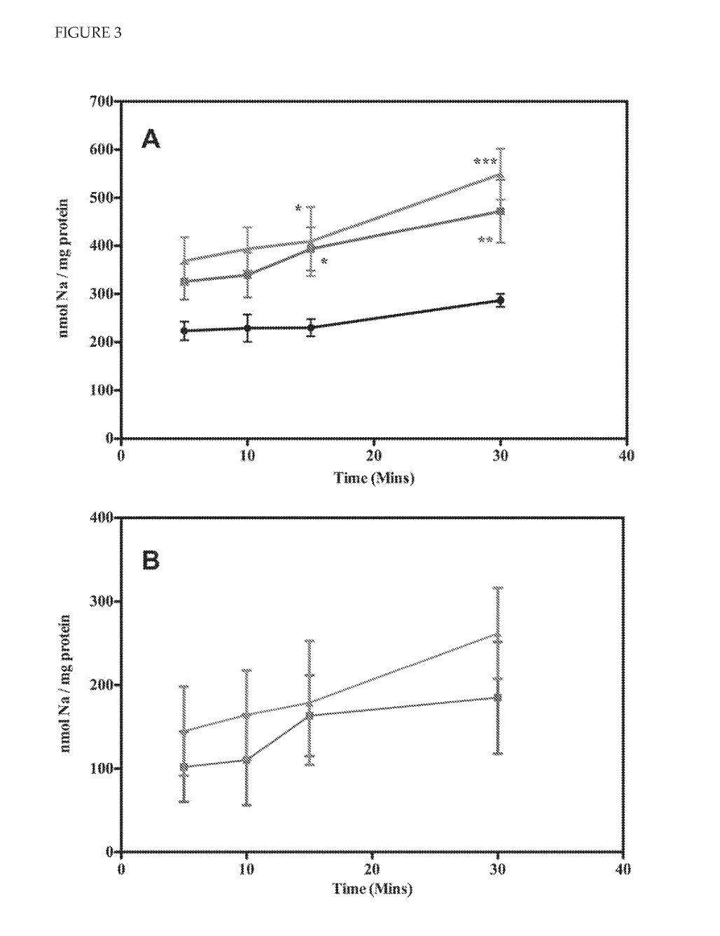

Investigation of ion selectivity in cells expressing either YDR352w or YOL092w was carried out using ²²Na flux analysis (FIGS. 3 A and B). Na⁺ flux was significantly greater in cells over expressing YDR352w and YOL092w when compared to the empty vector control cells.

### Example 3

### Characterisation of YDR352w and Yol092w Mediated Flux Through Expression in Xenopus laevis oocytes

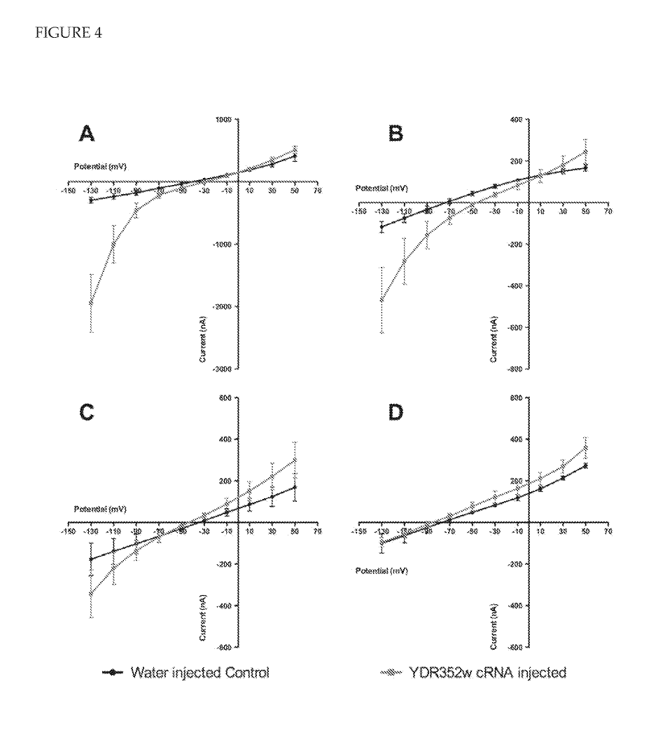

Xenopus laevis oocytes are useful for exploring the electrophysiology of membrane bound proteins. cRNA of YDR352w was used to optimise conditions for the analysis of cation flux through PQ loop repeat proteins. Initial experiments used choline-Cl as the predominant cation in the bath solution. This allows good current flow without being transported itself due to its size. These experiments revealed a strong induction of current in oocytes injected with YDR352w cDNA when voltage was clamped at hyperpolarising potentials (FIG. 4, panel A and FIG. 10, panel A). This is indicative of the native Xenopus Ca²⁺ activated Cl⁻ channel. The induction of this current suggests that either the expressed protein is inducing the native Ca²⁺ activated Cl⁻ channel or that YDR352w in facilitating the flux of Ca²⁺ into the oocyte and driving this Ca²⁺ activated Cl⁻ channel current. This current will interfere with observation of inward positive current and must therefore be minimised. To reduce potential sources of variation a minimalistic bath solution was used, 200 mM mannitol buffered to pH 7.0 with a minimal amount of Tris-Cl. With 1 mM CaCl₂ and 2 mM MgCl₂ in this basic buffer (FIG. 4, panel B and FIG. 10, panel B), currents resembling the Ca²⁺ activated Cl⁻ channel were present, although much reduced when compared to the choline-Cl based buffered traces. This indicates that either the higher external Cl⁻ concentration was inducing the Ca²⁺ activated Cl⁻ channel or choline itself was being fluxed through the protein and contributing to the total flux at negative membrane potentials or possibly a combination of both. Replacing Ca²⁺ with Ba²⁺ further reduced the difference between the YDR352w injected oocytes and the water injected oocytes (FIG. 4, panel C and FIG. 10, panel C), suggesting the presence of Ca²⁺ was inducing current flow. Removal of all divalent cations, leaving only 5 mM MES/Tris, effectively abolished any inward positive/outward negative ion flux at negative potentials (FIG. 4, panel D and FIG. 10, panel D). As a result of the low current flow observed at negative membrane potentials, the conditions under which inward cation flux may be observed, this buffer was chosen as a base for further experiments.

### Example 4

### Characterisation of Cation Flux in Xenopus laevis Oocytes Expressing YDR352w and YOL092w

Characterisation of transporters/channels in electrophysiology typically involves analysis of species affinity and identification of blockers. YDR352w and YOL092w were investigated using such methods. Na⁺, Choline and Ca²⁺ flux increased in X. laevis oocytes injected with either YDR352w or YOL092w cRNA when compared to water injected controls. Preliminary experiments also suggest MA⁺, NH₄⁺, and K⁺ flux is also modified when these proteins are expressed.

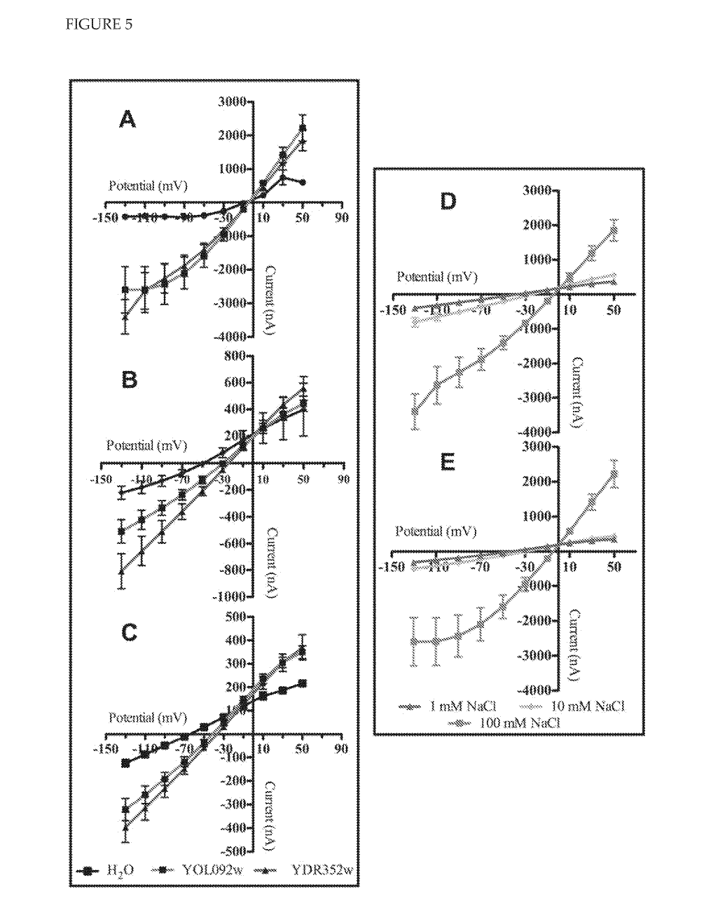

Of the cations used for testing expression in X. laevis, Na⁺ proved to be the most useful. Native currents in water-injected controls were relatively small and the proteins being examined elicited large Na⁺ induced currents. To ensure the currents observed were due to the influx of Na⁺ and not efflux of Cl⁻, buffers containing a series of buffers with differing NaCl concentrations were used (FIG. 5, panels A, B and C). As the Na⁺ concentration increases, the reversal potential (Eᵣₑᵥ) of the I/V curve plotted becomes more positive (FIG. 5, panels D and E), consistent with the influx of a cation as opposed to the efflux of the Cl⁻ anion.

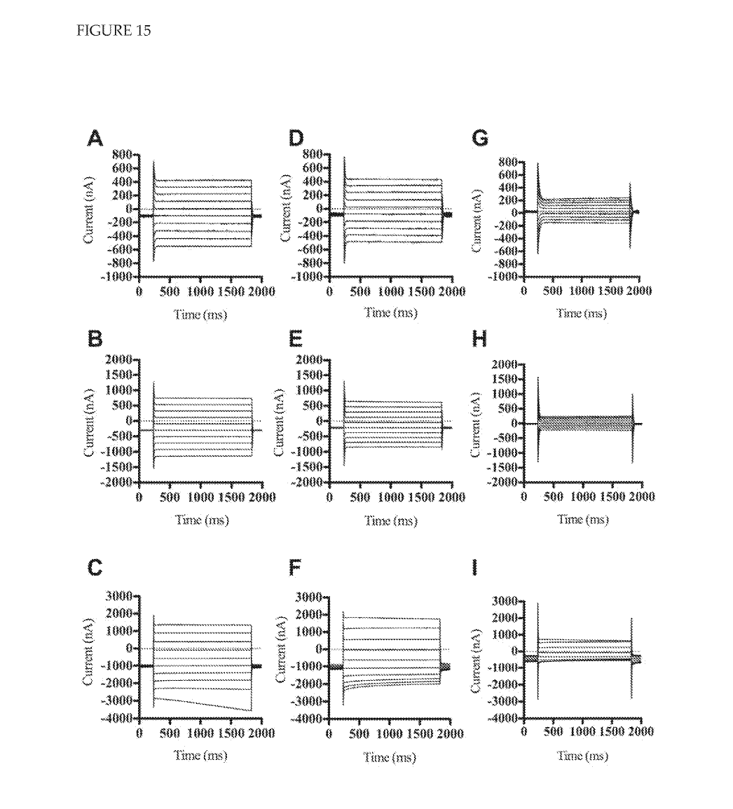

Significantly higher flux was recorded for both inward and outward cation flux when compared to the water injected control for all concentrations of Na⁺ examined (FIG. 5, FIG. 15). Whereas inward current has been established as being the result of Na⁺, cation efflux observed at positive membrane potential may not be due to Na⁺ flux. It is most likely that the observed flux is due to K⁺ movement, as this is the dominant monovalent cation within the oocyte. Similar increased outward flux is seen in YDR352w injected oocytes when voltage is clamped at positive potentials in the base buffer (FIG. 4, panel D and FIG. 10, panel D). This is consistent with these PQ loop repeat proteins facilitating the bi directional flux of cations.

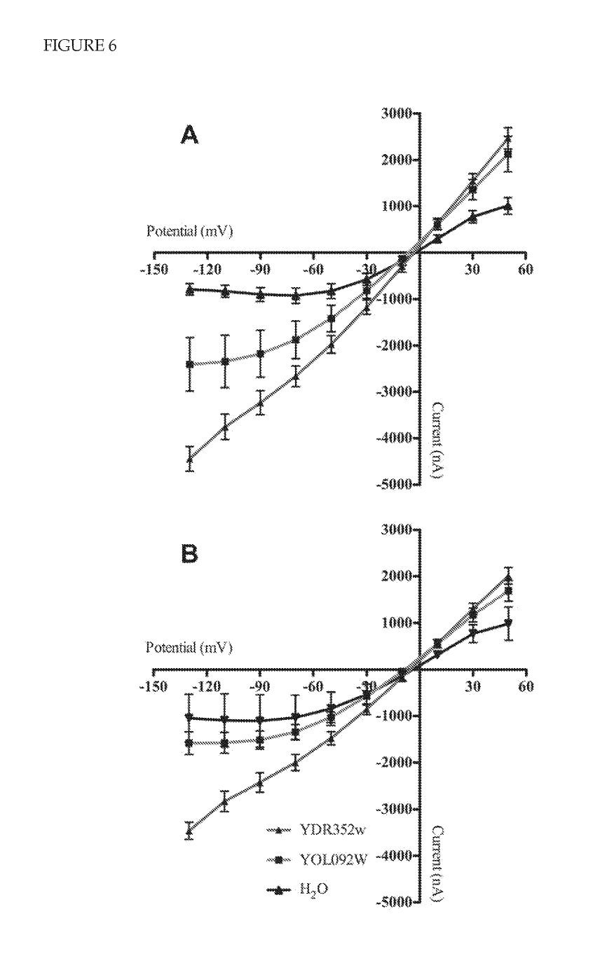

K⁺ is often the dominant cation fluxed in yeast and Xenopus oocyte expression systems and can sometime facilitate the flux of other cations. It was therefore relevant to investigate the influence of the native K⁺ transport systems on flux recorded from these PQ loop repeat proteins. TEA⁺ was used in its role as a K⁺ channel blocker to this end. The addition of 10 mM TEA⁺ to a bath solution containing 90 mM NaCl did not alter the general trends of flux but did influence current magnitude (FIG. 6). These data suggest that either TEA⁺ is being carried by YOL092w and YDR352w or that TEA⁺ is acting as an agonist to the NSCC induced traces, increasing their Na carrying capacity. The reversal potentials do not significantly change with the addition of TEA⁺, suggesting TEA⁺ itself is not being carried. It is possible that any difference in Eᵣₑᵥ is eclipsed by presence of much higher Na⁺ and internal K⁺ concentrations. The possible flux of TEA⁺ is intriguing given the structural similarities between TEA⁺, MA and NH₄⁺ and observed MA and NH₄⁺ flux characteristics (FIGS. 1, 2 and 7).

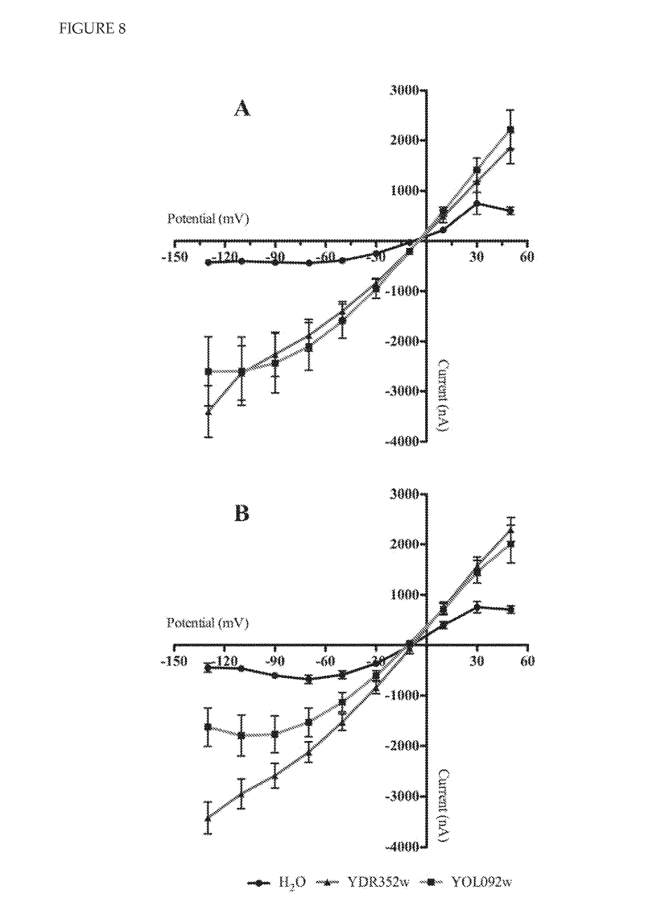

The influence of Cl⁻ on observed currents was investigated through the substitution of NaCl with Na₂SO₄ (FIG. 8). Currents were unchanged for oocytes expressing YDR352w, suggesting the gross currents were not influenced by external Cl⁻ concentration. Currents for YOL092w were decreased with the substitution of Cl⁻ for SO₄²⁻. This may be due either to a decrease in a Cl⁻ induced effect or as a response to added SO₄²⁻.

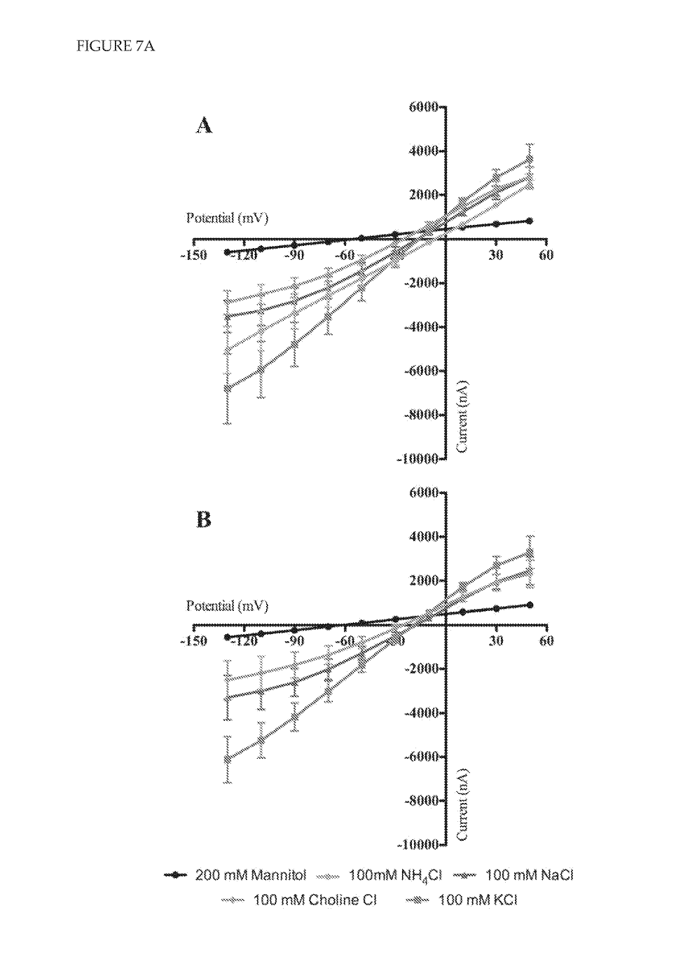

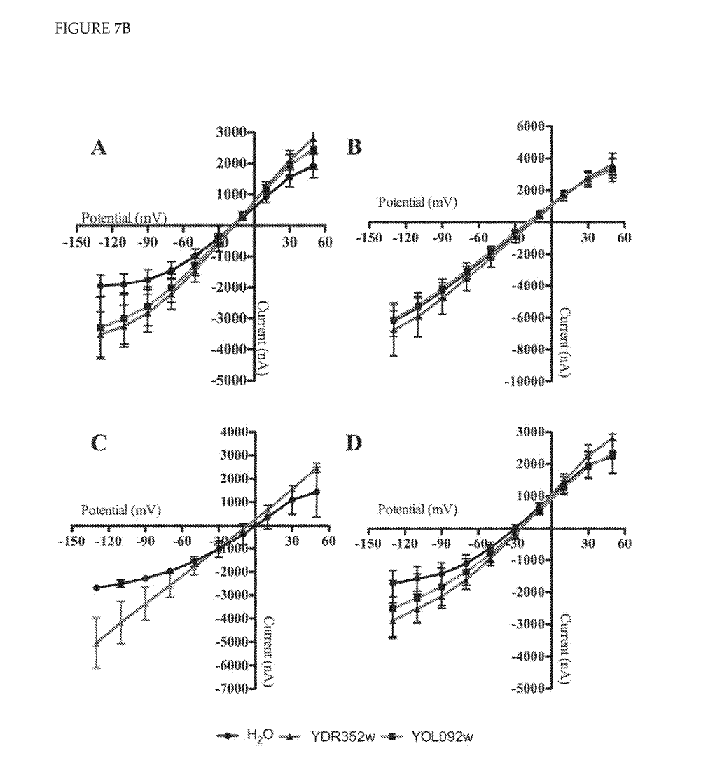

Analysis of cation selectivity suggests these PQ loop repeat proteins have poor discrimination between monovalent cations. Flux of choline⁺, Na⁺, and NH₄⁺ is increased in oocytes expressing YDR352w or YOL092w (FIG. 7).

Control oocytes showed a high degree of K⁺ flux, which masked any potential K⁺ flux through the PQ loop repeat proteins examined. NH₄⁺ flux was measured in oocytes injected with YDR352w and H₂O only. With the data available there is a strong suggestion that YDR352w facilitates NH₄⁺ flux.

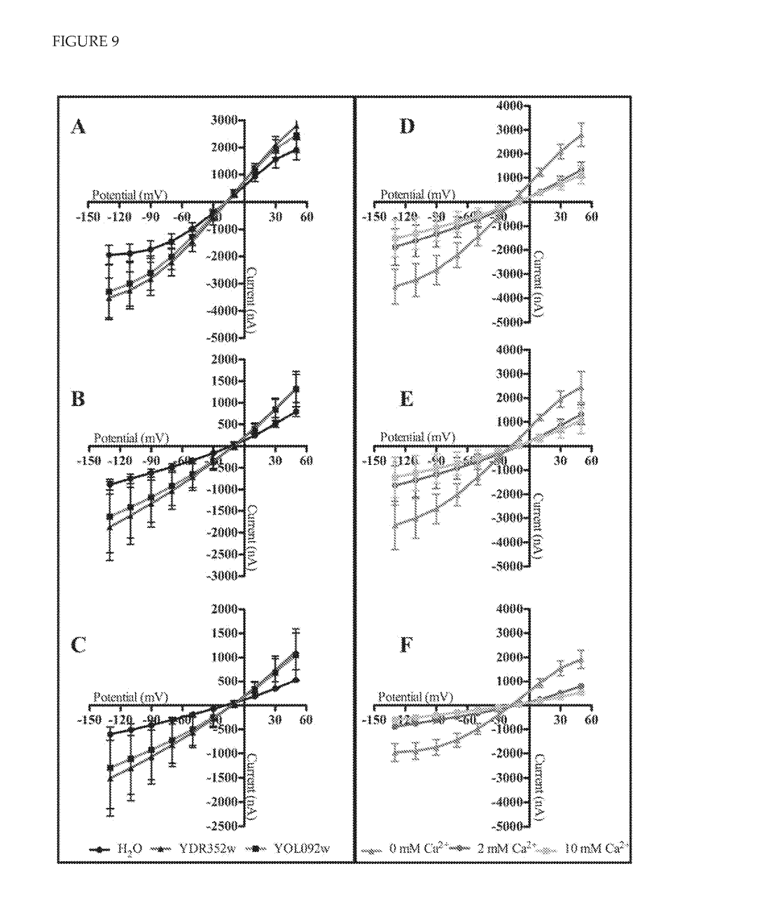

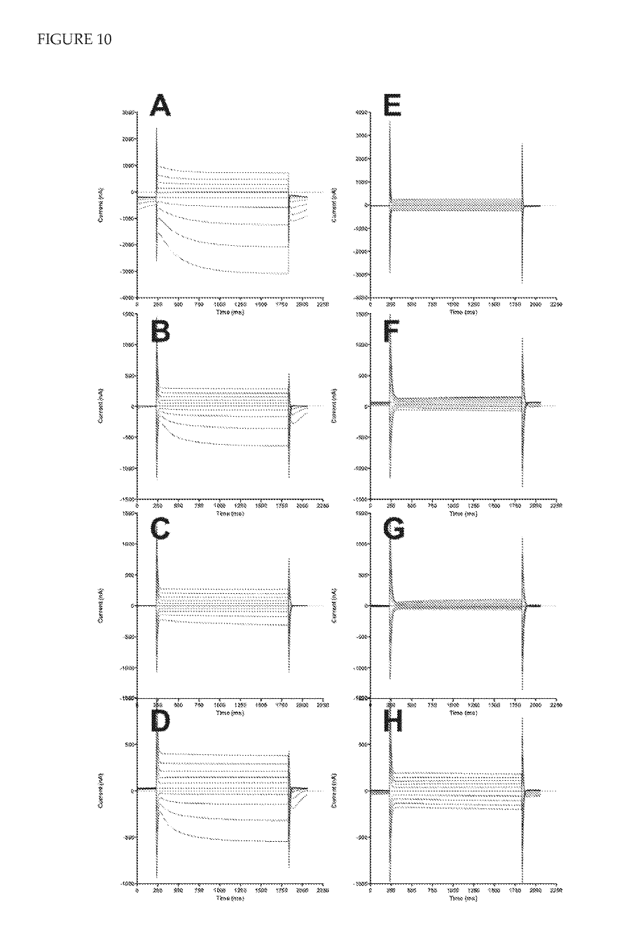

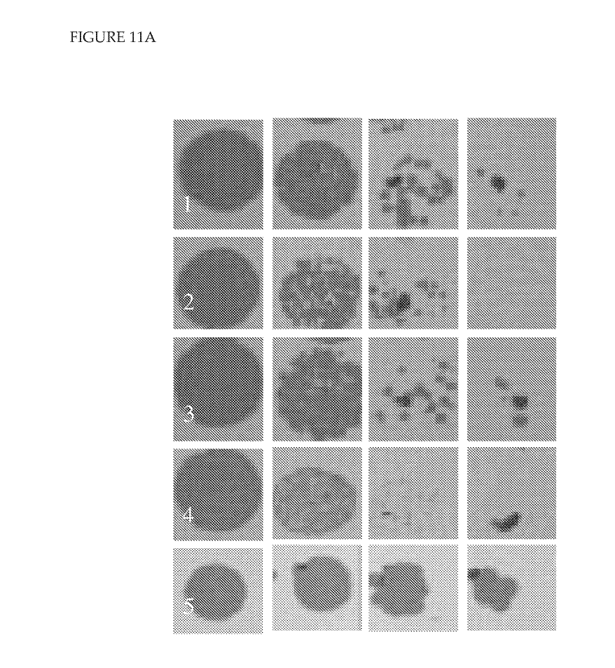

The effect of differing Ca²⁺ concentrations on the flux of Na⁺ in oocytes expressing YDR352w and/or YOL092w was also investigated (FIG. 9). A significant decrease in both inward and outward current was recorded with the addition of 2 mM CaCl₂ and more still with the addition of 10 mM CaCl₂.

### Example 5

¹⁴C Labelled MA Flux and ²²Na Labelled Na⁺ Flux Analysis

A pDONR (Invitrogen) vector containing either YDR352w or YOL092w was used to recombine the insert of interest into the vector pYES2-DEST using the LR clonase reaction (Invitrogen). Cells containing YDR352w or YOL092w in pYES3-DEST or empty vector were grown to saturation in liquid YNB supplemented with 2% D-Glucose (w/v), harvested via centrifugation at 4000×g for 4 minutes and used to inoculate Grensons liquid media at pH 6.5 with 0.1% L-proline and 2% D-Galactose (w/v) to OD₆₀₀=0.1. Cells were incubated overnight at 28° C. shaking at 200 rpm and harvested at OD₆₀₀=0.4−0.7 by centrifugation at 4000×g, washed twice in MilliQ H₂O and resuspended in 20 mM KPO₄⁻ buffer pH 6.5 with 2% (w/v) D-Galactose to give OD₆₀₀˜4-6. A stock solution of 1 M MACl or NaCl was added to a 20 mM KPO₄⁻ buffer with at pH 7.0 to the concentration required and labelled with either ¹⁴C MA (Amersham) or ²²Na (Amersham). This was added to an equal volume of resuspended cells at t=0 and were shaken continuously throughout the flux experiment. At the specified time, samples were removed, passed through a 0.45 μM nitrocellulose filter (Whatman) and washed with 10 ml of ice-cold 20 mM KPO₄⁻ buffer to cease flux. Membranes were collected, placed in a 7 ml scintillation vial (Sarstedt) and 4 ml scintillation fluid was added (Perkin Elmer). Samples were counted in a liquid scintillation counter (Packard). Counts were converted into equivalent amount of MA⁺ or Na⁺ and samples were normalised against total protein, derived from a modified Lowry method (Peterson, Analytical Biochemistry 83: 346-356, 1977).

### Example 6

### Synthesis of cRNA and Injection into X. laevis Oocytes

Oocytes of Xenopus laevis were prepared as per standard protocols (Zhou et al., Plant, Cell & Environment 30: 1566-1577, 2007) with the use of Calcium Frog Ringers solution (96 mM NaCl, 2 mM KCl, 5 mM MgCl₂, 5 mM HEPES, 0.6 mM CaCl₂) plus 8% horse serum, 0.1 mg/ml Tetracycline, Penicillin 1000 u/ml and Streptomycin 0.1 mg/ml). A pDONR (Invitrogen) vector containing either YDR352w or YOL092w was used to recombine the insert of interest into the vector pGEMHE using the LR clonase reaction (invitrogen). The pGEMHE vector containing the gene of interest was digested overnight with Sph1 (NEB) to linearise the DNA. The mMessage mMachine 5′ capped RNA transcription kit (Ambion) was used according to the manufacturer's protocol to synthesise cRNA for the gene of interest. cRNA concentration was normalised to 1 μg/μL and 46 nL injected into each oocyte using a micro injector (Drummond ‘Nanoject II’ automatic nanolitre injector, Drummond Scientific, Broomall, Pa., USA). Control oocytes were injected with 46 mL of nuclease free H₂O. Injected oocytes were incubated at 16° C. in Calcium Frog Ringers solution for 3 days prior to use.

### Example 7

### Electrophysiology of S. Cerevisiae PQ Loop Repeat Proteins Expressed in Xenopus laevis Oocytes

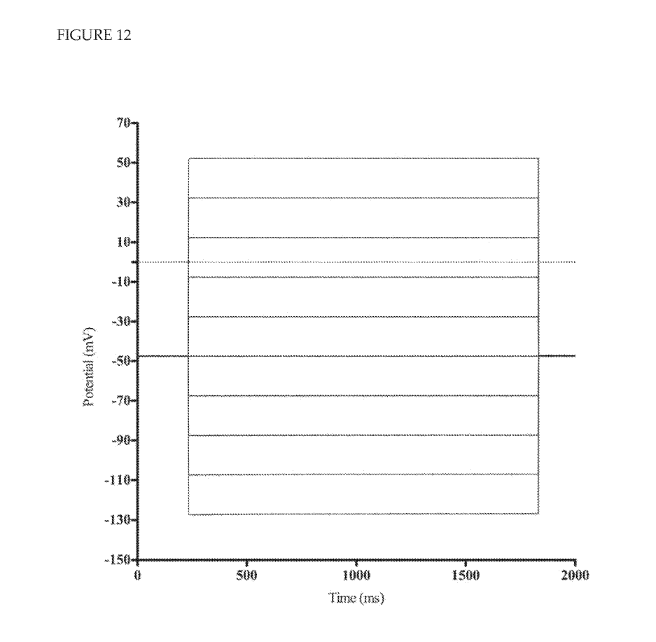

Unless otherwise specified, oocyte voltage clamping occurred at 25° C. in a base buffer consisting of 5 mM MES/Tris at pH 7.0, with ions of interest added. Mannitol was used to keep osmolarity at 200 mOsm. Signal was amplified using a Gene Clamp 500 voltage clamp amplifier (Axon Instruments, Molecular Devices, Sunnyvale, Calif., USA) and displayed using Clampex 8.2 (Axon Instruments). Oocytes were impaled with glass capillaries filled with 3 M KCl. Electrodes responsible for maintaining the voltage clamp and current flow were bathed in this 3 M KCl solution. The voltage protocol used was as shown in FIG. 12.

### Example 8

### Discussion

Voltage insensitive NSCCs were initially considered to be ‘leak’ currents when observed in patch clamping experiments. Observation of channel mediated current through these proteins often require a low external Ca²⁺ concentration and were subsequently thought to be the result of a loss of membrane integrity. Detailed investigation ascertained that monovalent cation flux was favoured over divalent cation flux and thus the presence of the protein deduced.

Since their discovery, the genetic identity of voltage insensitive NSCCs has been sought. Currents with viNSCC properties have been recorded in the ‘pacemaker’ sinoatrial node cells of rabbit hearts and could play an important role in regulating heartbeat. Similar currents have also been recorded in, for example, Xenopus oocytes, many plant species and yeast.

As these proteins catalyse high capacity monovalent cation flux, they have the potential to significantly impact cell function. It is this potential that makes viNSCCs strong candidates for the large Na⁺ flux observed in plants when they are exposed to saline conditions.

Until now, the molecular identities of viNSCCs have been unknown. This is likely due to difficulties in using heterologous screens as viNSCCs passively catalyse ion flux and hence show subtle phenotypes.

Both ¹⁴C labelled MA⁺ and ²²Na labelled flux suggested that YDR352w and YOL092w were cation channels. ¹⁴C labelled MA flux showed a consistently higher rate of MA flux in the LATS range for both YDR352w and YOL092w over expressing cells when compared to the empty vector control (FIG. 1). A concentration profile showed these proteins exhibit unsaturable MA uptake kinetics well into the LATS range of yeast (FIG. 2). ²²Na⁺ flux experiments mirrored the MA⁺ flux data by showing an increased rate of Na⁺ influx into cells over expressing YDR352w or YOL092w compared to empty vector transformed control cells (FIGS. 3 A and B).

The increased capacity of cation influx observed in cells over expressing both YDR352w and YOL092w is consistent with that expected for an over expressed viNSCC. Flux at both 50 mM MA⁺ and 50 mM Na was greater than that of the empty vector control. Flux only becomes significantly different from the empty vector control after 5 minutes of flux with 50 mM MA⁺ (FIG. 1) or after 15 minutes with 50 mM NaCl (FIGS. 3 A and B). Similarly, resolution between the empty vector control and candidate proteins in the MA concentration profiles was only obtained at 25 mM for YDR352w and 50 mM for YOL092w. Native yeast proteins that flux either MA or Na⁺ (or both) are active in this strain. This includes YDR352w and YOL092w, our candidate NSCCs. Empty vector controls will therefore behave in a manner similar to those transformed with the candidate genes, the difference being candidate protein over expression in the transformed cells. Poor resolution is expected and observed as a result, especially for Na⁺ flux as all native Na⁺ transporters are still present. Resolution for MA flux is likely to be better than for Na⁺ as the native MEPs have been deleted from this strain. Nonetheless, significant differences in MA and Na⁺ flux profiles are observed in the candidate gene transformed cells when compared to the control cells.

Expression of YDR352w and YOL092w cRNA in Xenopus significantly increased current flow in oocytes in Na⁺ containing bath solution. Current amplitude was influenced by Na⁺ concentration with greater current observed at higher Na⁺ concentrations (FIG. 5). The greater the Na⁺ concentration, Eᵣₑᵥ moved positive. This confirms the current recorded was the result of Na⁺ flux as opposed to Cl⁻. Confirmation of this trait was achieved through the substitution of 100 mM NaCl with 50 mM Na₂SO₄ (FIG. 8) revealing little difference in Eᵣₑᵥ.

Both YDR352w and YOL092w exhibit similar physiology both when over expressed in yeast and when expressed in Xenopus oocytes. This is not surprising as they share many sequence and predicted structural traits. Subtle differences are evident within these data suggesting discreet roles for each protein. These proteins show differences when in a hyperpolarised membrane. The current/voltage relationship YDR352w shows in a Ca²⁺ free Na bath solution is reasonably linear for both proteins across the range of potentials investigated. YOL092w, however, shows a loss of linearity at potentials of −70 mV and less, mirroring the response shown in water injected control oocytes.

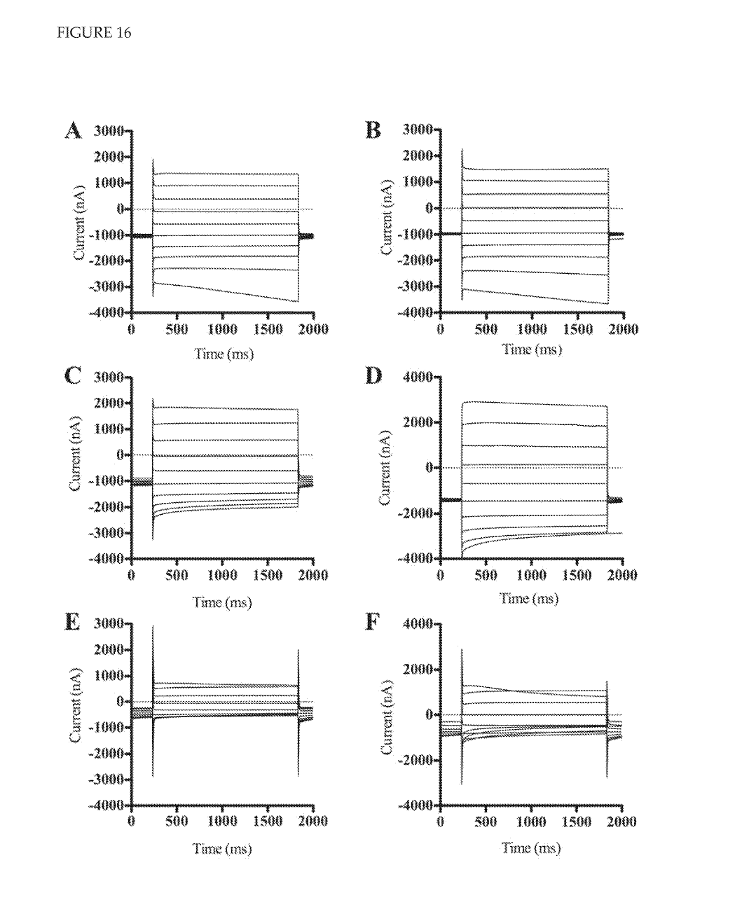

Currents observed showed different degrees of time dependence. Overall, currents elicited through YDR352w showed little time dependence (FIG. 10 panels A, B and C and FIG. 15, panels A, B and C). Some time dependence was observed at low membrane potentials when SO₄²⁻ is used as the Na⁺ coupled anion (FIG. 16, panels A and B). As this is not seen when Cl⁻ is the counter ion and differences are also seen in both YOL092w (FIG. 16, panels C and D) and water injected oocytes (FIG. 16, panels E and F), it may be an artefact of SO₄²⁻ presence in the bath solution. YOL092w elicited currents were mostly independent of time, except when 100 mM Na⁺ was present in the bath solution (FIG. 15, panels D, E and F). Under these conditions, currents obtained at potentials of −90 mV and less show a time dependant decrease until a steady state is reached after approximately 1.5 seconds. Current measurements for the generation of I/V curves were taken as the average of current from 1.2 to 1.6 seconds into the voltage clamp.

Changes in Ca²⁺ activity influenced the current due to Na⁺ flux and slightly altered Eᵣₑᵥ (FIG. 9). A slight positive shift of Eᵣₑᵥ is present with increasing Ca²⁺ concentration in the bath solution, suggesting that Ca²⁺ may flux through these proteins. The observed activation of the Ca²⁺ activated Cl⁻ in oocytes expressing YDR352w (FIG. 4, panel A and FIG. 10, panel A) also suggests Ca²⁺ flux is increased. Current was reduced by approximately 50% in all traces upon the transition from 0 mM Ca²⁺ bathing solution to a 2 mM Ca²⁺ bathing solution. Current was reduced by a further 20% when the Ca²⁺ concentration was increased from 2 mM to 10 mM. Overall, the addition of 10 mM Ca²⁺ to a previously Ca²⁺ free bath solution reduces current by approximately 60%.

Weak discrimination between monovalent cations is the defining characteristic of viNSCCs. YDR352w and YOL092w were investigated in terms of their monovalent cation channel selectivity when expressed in Xenopus laevis oocytes (FIGS. 7 A and B). Choline⁺ and Na⁺ flux is catalysed by the presence of either YDR352w or YOL092w. Preliminary data strongly suggests that K⁺, NH₄⁺ and MA⁺ fluxes are also increased when these genes are expressed (FIGS. 1, 2 and 7). The induction of the Ca²⁺ activated Cl⁻ channel, combined with the inhibitory effect Ca²⁺ has on Na⁺ flux, also suggests that Ca²⁺ flux is also facilitated by these proteins.

### Example 9

### Characteristics of PQ Loop Repeat Proteins

Solid media growth phenotypes (FIG. 11, panels A, B and C) and ¹⁴C labelled MA⁺ flux data (FIGS. 1 and 2) suggest YDR352w and YOL092w are putative viNSCCs. Both of these proteins belong to the PQ loop repeat class of proteins. The yeast PQ loop proteins selected are therefore most likely viNSCC candidates. YDR352w and YOL092w, as well as the very similar YBR147w, have no known biological or molecular function recorded in the Saccharomyces Genome Database (SGD). YBR147w was not selected by the genome database search as it is not predicted to be membrane associated. This is likely erroneous due to its similarity to other PQ loop repeat proteins.

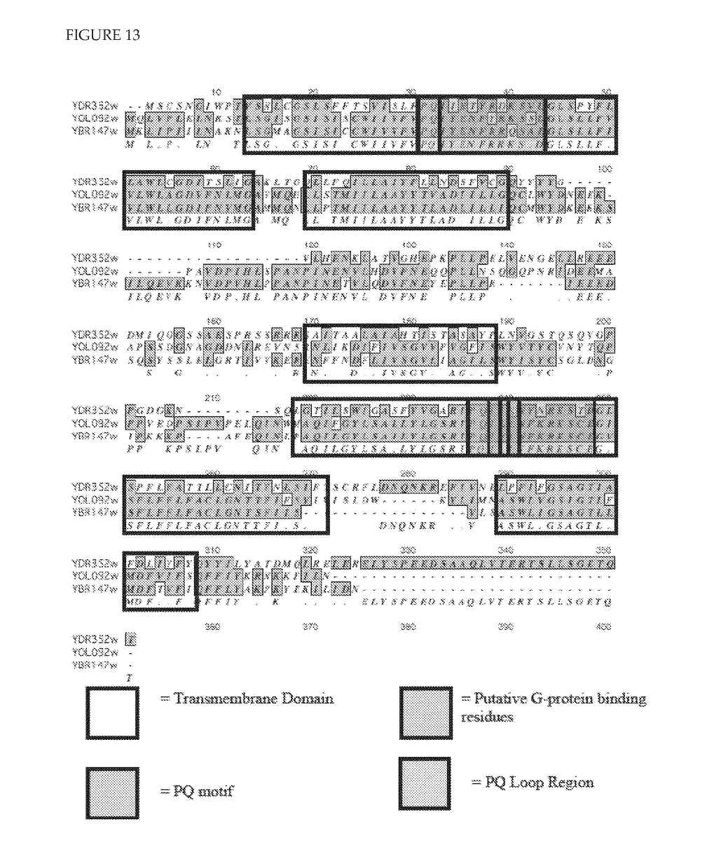

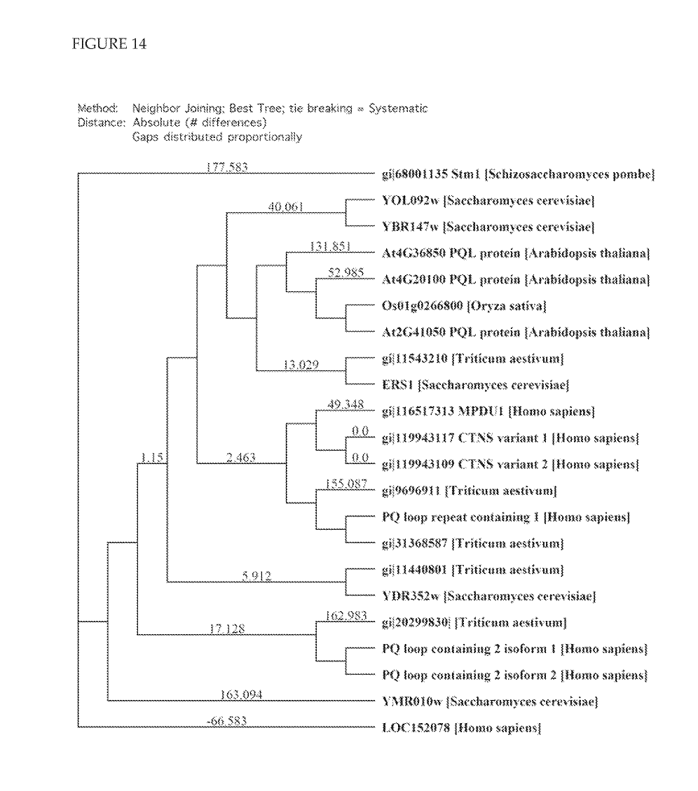

PQ loop repeat proteins are characterised by repeats of a proline and glutamine (PQ) residues prior to an extra membrane loop (FIG. 13). They are largely unannotated. Examples of characterised PQ loop repeat proteins are the human CTNS and MPDU1, the S. cerevisiae ERS1 and stm1+ from Schizosaccharomyces pombe. CTNS and ERS1 transport L-cystine across biological membranes. ERS1 deletion mutants in yeast have also been implicated in the disruption of protein retention in the ER. HsMPDU1 has been associated with a ‘flippase’ mechanism involving lipid linked oligosaccharides. Stm1 has been shown to interact with a G-protein and is subsequently designated as a G-protein coupled receptor.

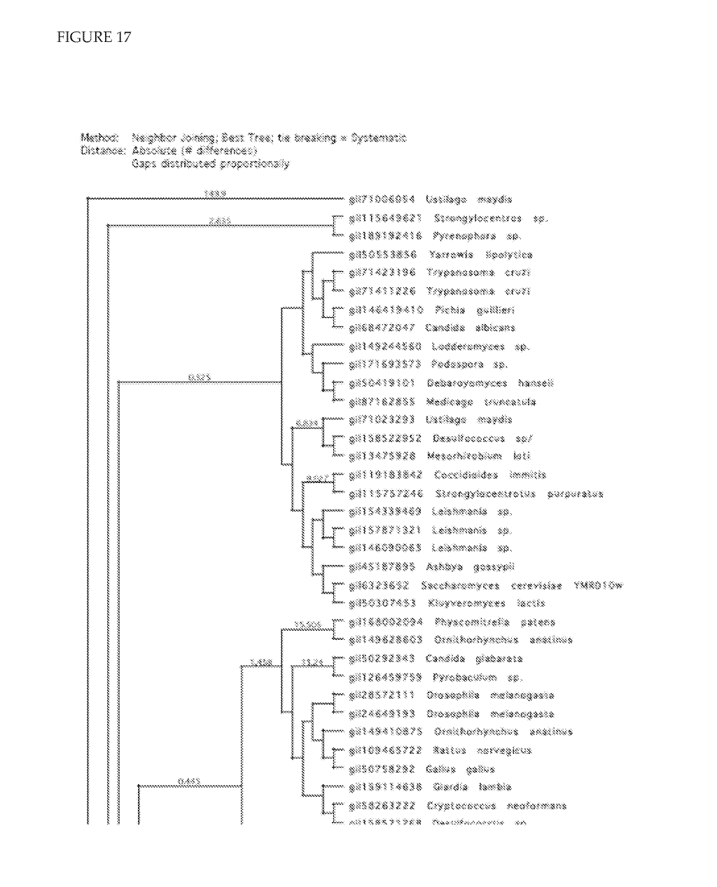

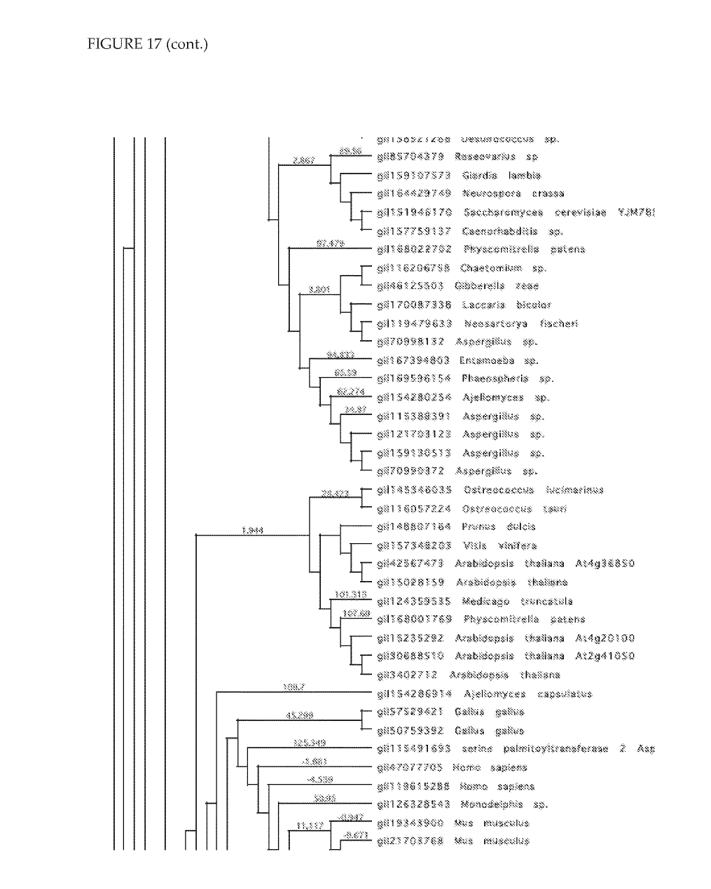

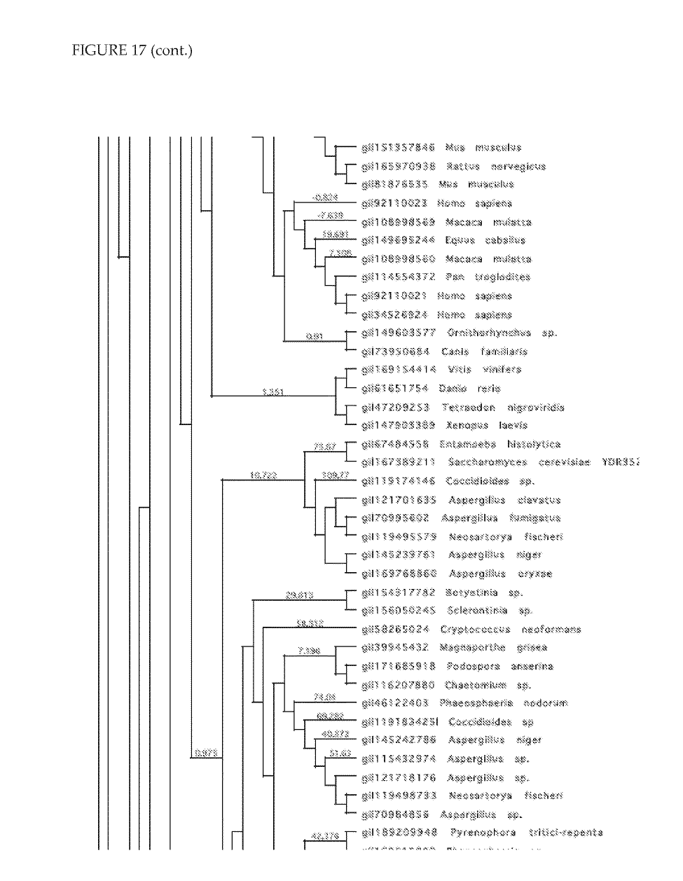

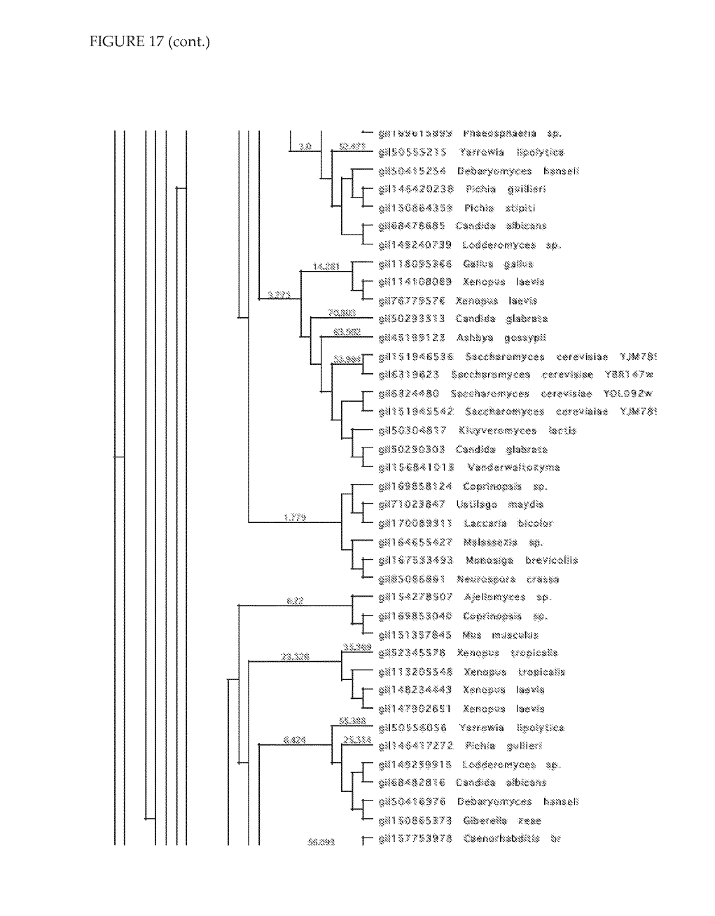

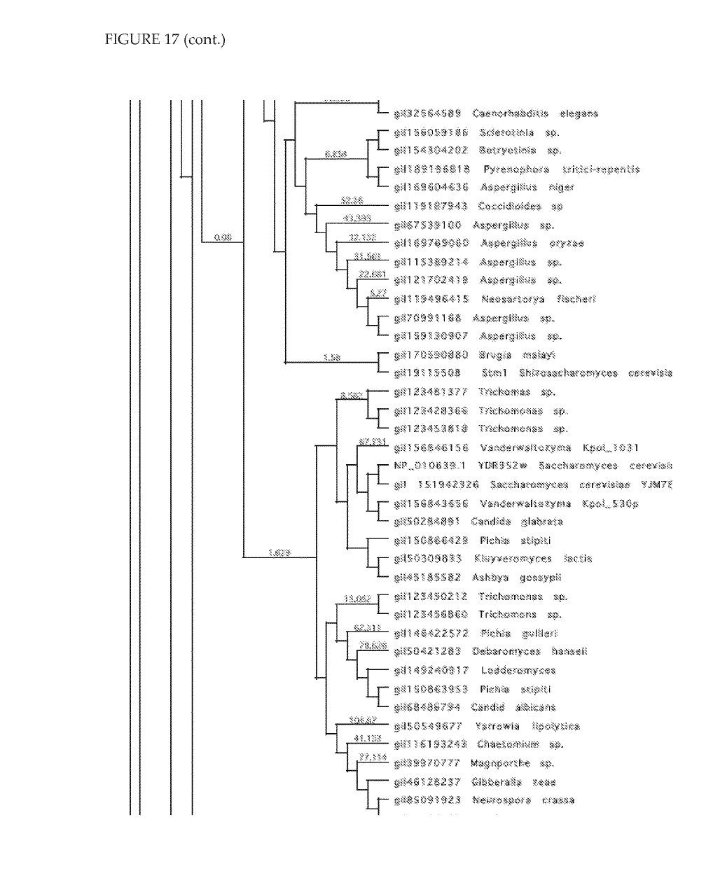

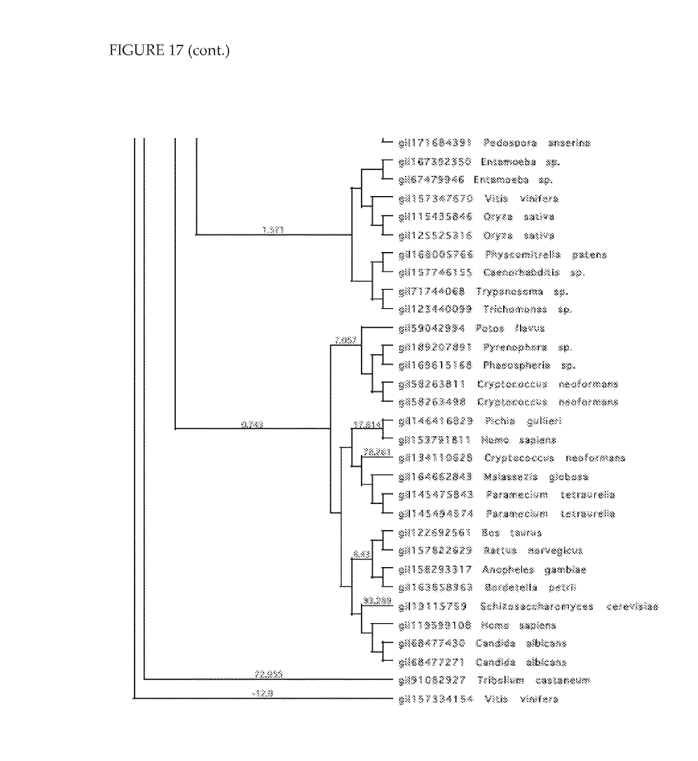

Some structural information has been obtained from investigated PQ loop proteins (FIG. 13). Loop 2 of the PQ loop motif may be important in protein localisation, as shown with HsCTNS. GFP localisation of other PQ loop proteins has shown they are integrated in a variety of membranes with loop 2 shown to be vital for correct protein trafficking. The third predicted cytoplasmic domain of Stm1 is predicted to be the site of G-protein interaction. This is of particular importance to any signalling cascades within which these proteins may be involved. BLAST searches of YDR352w across available genome databases revealed many similar proteins (FIG. 17), most of which are also unannotated with the exception of those mentioned previously.

Expression profile data is available for yeast ORFs through SGD. Investigation of these data can give some insights into the function of the proteins. Transcription of all three genes of interest is influenced by yeast nutritional status. Specifically, strong changes are recorded for yeast that is well into stationary phase of growth, sporulation, N depletion and Ca²⁺/Na⁺/calcineurin responses. These responses indicate a possible link with ion sensing or ion transport within the cell.

### Example 10

### Materials and Methods

### Growth Phenotypes on Solid Media of S. cerevisiae Strain 31019b Overexpressing Candidate Genes

Vectors with candidate genes were sourced from the Open Biosystems Yeast ORF Collection. They consist of individual yeast ORFs cloned into the vector BG1805. Each ORF is expressed under the high expression Gall promoter and has had its stop codon removed to incorporate a C-terminal HA protein tag (Gelperin et al., Genes and Development 19: 2816-2826, 2005). Each clone was transformed into 31019b (Marini et al., Mol. Cell. Biol. 17: 4282-4293, 1997) using the lithium acetate/poly ethylene glycol method (Gietz et al., Yeast 11: 355-360, 1995) and transformants selected on YNB minimal media. Transformed strains were individually grown overnight in liquid yeast nutrient base (BD biosciences, San Jose, USA; 0.67% (w/v), D-glucose 2% (w/v) pH 6.5) to late log phase. Cells were pelleted and washed twice in sterile milliQ water and re-suspended to OD₆₀₀ of 0.3. Cultures were serially diluted to a final OD₆₀₀ of 0.003 and 5 μL of each dilution placed on solid yeast minimal media (Grenson, Biochimica et Biophysica Acta 127: 339-346, 1966) with 0.1 M MA, 0.1% (w/v) L-proline, 2% (w/v) D-galactose at a pH of 7.0 with either 0.2 mM Ca²⁺ or 10 mM Ca²⁺. Plates were incubated at 28° C. for 5-7 days and growth phenotypes monitored. Plasmids which, when expressed, induced phenotypes indicative of increased MA⁺ toxicity in cells were selected and analysed further.

### Example 11

### Plant PQLs

### Materials and Methods

To investigate the effect on shoot Na⁺ accumulation AtPQL1 (At4g20100), AtPQL2(At2g41050) and AtPQL3(At4g36850) were constitutively expressed in Arabidopsis (Table 2). To investigate the effect on shoot Na⁺ accumulation OsPQL1 (Os01g16170) was constitutively expressed in rice (Table 2). AtPQL1, AtPQQL2 and AtPQL3 were also heterologously expressed in yeast.

TABLE 2
Names and accession numbers of Arabidopsis and rice PQL proteins

| | | | No. | | Position |
|---|---|---|---|---|---|
| | | UniProt | Of AA | Position of | of 2^{nd} |
| Name | Accession | Accesion | Residues | 1^{st} PQ-loop | PQ-loop |
| AtPQL1 | At4g20100 | O49437 | 288 | 3-69 | 187-242 |
| AtPQL2 | At2g41050 | Q8RXY4 | 376 | 8-74 | 268-332 |
| AtPQL3 | At4g36850 | O23198 | 374 | 30-96 | 255-321 |
| AtPQL4 | At5g59470 | Q9LTI3 | 239 | 27-93 | 150-205 |
| AtPQL5 | At5g40670 | P57758 | 270 | 9-75 | 151-213 |
| AtPQL6 | At4g07390 | Q8VY63 | 234 | 27-93 | 150-205 |
| OsPQL1 | Os01g16170 | Q5NBM2 | 421 | | |
| OsPQL2 | Os07g29610 | Q7X990 | 244 | | |
| OsPQL3 | Os12g18110 | Q2QTY6 | 274 | | |

DNA and RNA Extractions and cDNA Synthesis

Genomic DNA was extracted from young leaves of Arabidopsis thaliana using the methodology of Edwards et al. (Nucleic Acids Research 19: 1349, 1991). Briefly, plant shoot or root tissue was snap frozen in liquid nitrogen and ground to a fine powder using a mortar and pestle. To the powder, 400 μl of Edwards buffer (200 mM Tris pH 8, mM EDTA, 250 mM NaCl and 0.5% SDS), was added and the samples left at room temperature for 1 hr. The samples were centrifuged at 13,000 g for 2 mins and the supernatant removed. DNA was precipitated by the addition of 300 μl of 100% isopropanol, incubation of the samples at room temperature for 2 mins, before centrifugation at 13,000 g for 5 mins. DNA pellets were washed with 70% ethanol and allowed to air dry before being resuspended in 100 μl of TE buffer.

Total RNA was extracted using TRIzol reagent (Invitrogen, Carlsbad, Calif., USA), following the protocol described by (Chomczynski, BioTechniques 15: 532-537, 1993). Genomic DNA contamination was removed using Ambion's DNA free (Ambion, Madison, Wis., USA) and 2 μg of total RNA was used to synthesis cDNA using Superscript III (Invitrogen).

### Over-Expression of AtPQL1 to 3 in Arabidopsis

Transgenic Arabidopsis plants constitutively expressing the genes AtPQL1, AtPQL2 and AtPQL3 using a 35S promoter, were obtained from Dr Anna Amtmann, University of Glasgow, UK. Using primers AtPQL1, 2 or 3 Whole gene Forward and AtPQL1, 2 or 3 Whole gene Reverse (Table 3), the complete gene was cloned from Arabidopsis Col-0 genomic DNA into a pCR8 Gateway enabled entry vector. Restriction digestion and sequencing of the plasmid was used to confirm the orientation of the gene in the vector and to ensure there were no errors in the coding sequence of the cloned gene. For Arabidopsis transformation, the gene was then transferred into a pGWB2 destination vector, using a Gateway reaction, and transformed into Agrobacterium tumefaciens, strain GV3101.

TABLE 3
Sequences of primer pairs used to amplify the
complete gene of AtPQL 1, 2 and 3 and OsPQL1

| Gene | Forward | Reverse |
|---|---|---|
| AtPQL1 | ATGATTCGAGATGATTTGTC | TTAGACAGCTTCTTCACCGG |
| | (SEQ ID NO: 10) | (SEQ ID NO: 11) |
| AtPQL2 | ATGTTTCTCCACAGCAGTTT | TTAGACAGCTTCTGCGGTT |
| | (SEQ ID NO: 12) | (SEQ ID NO: 13) |
| AtPQL3 | ATGGATTATCTAGGAATCGA | TGAAATCTTTTTACTTTTCA |
| | (SEQ ID NO: 14) | (SEQ ID NO: 15) |
| OsPQL1 | ATGGGCATCTTCAGTGGAGC | TTAAACTTTATCAGCACTGTC |
| | (SEQ ID NO: 16) | (SEQ ID NO: 17) |

### SALK T-DNA Knockout

All T-DNA knockout mutants were obtained from the SALK collection via the Nottingham Arabidopsis Stock Centre (NASC, Nottingham, UK). To select homozygotes, plants were grown individually on soil and their zygosity tested by genomic DNA isolation and PCR, using primers designed by the Signal iSect tool (signal.salk.edu/tdnaprimers.2.html) to detect the T-DNA insert. Transcript levels of the knockout lines pql1-1 (SALK_{—}108796) and pql1-1 (SALK_{—}044346) were checked by semi-quantitative reverse transcription PCR.

amiRNA Knockdowns

As AtPQL1 to 3 share a high homology, it may be possible that there is redundancy in the gene family. To check this, amiRNA mutants were designed to knockdown 2 or 3 of AtPQL1-3 genes at the same time. WMD 2—Web MicroRNA Designer (http://wmd2.weigelworld.org/cgi-bin/mimatools.pl) was used to identify two 21 base sequences to which two independent amiRNA construct could be designed which would reduce the expression of either AtPQL1&2, AtPQL1&3, AtPQL2&3, or AtPQL1,2&3. Primers (Table 4) containing the necessary sequences to generate 21 bp amiRNAs were incorporated into the amiRNA vector MTR319a and the whole amiRNA constructs were cloned into pCR8, following the protocol at http://wmd2.weigelworld.org/cgi-bin/mimatools.pl?page=7. After sequencing, to check for any sequence errors and to determine the correct orientation of the sequence, a Gateway LR was performed to transfer the amiRNA constructs into pTOOL2 vectors which would use a 35S promoter to drive the expression of the amiRNA.

TABLE 4
amiRNA target sequences and primers

| Name | AtPQL target sequence | Primer Name | Sequence |
|---|---|---|---|
| amiRNA | TATTAATGGATTCTATCCCTC | I miR-s | GATATTAATGGATTCTATCCCTCTCTCTCT |
| AtPQL1&2 | (SEQ ID NO: 50) | | TTTGTATTC |
| #1 | | | (SEQ ID NO: 18) |
| | | II miR-a | GAGAGGGATAGAATCCATTAATATCAAAGA |
| | | | GAATCAATGA |
| | | | (SEQ ID NO: 19) |
| | | III miR*s | GAGAAGGATAGAATCGATTAATTTCACAGG |
| | | | TCGTGATATG |
| | | | (SEQ ID NO: 20) |
| | | IV miR*a | GAAATTAATCGATTCTATCCTTCTCTACAT |
| | | | ATATATTCCT |
| | | | (SEQ ID NO: 21) |
| amiRNA | TAGTTAGTCATAATCTACGGT | I miR-s | GATAGTTAGTCATAATCTACGGTTCTCTCT |
| AtPQL1&2 | (SEQ ID NO: 51) | | TTTGTATTCC |
| #2 | | | (SEQ ID NO: 22) |
| | | II miR-a | GAACCGTAGATTATGACTAACTATCAAAGA |
| | | | GAATCAATGA |
| | | | (SEQ ID NO: 23) |
| | | III miR*s | GAACAGTAGATTATGTCTAACTTTCACAGG |
| | | | TCGTGATATG |
| | | | (SEQ ID NO: 24) |
| | | IV miR*a | GAAAGTTAGACATAATCTACTGTTCTACAT |
| | | | ATATATTCCT |
| | | | (SEQ ID NO: 25) |
| amiRNA | TCGCCATGTAAATCGCGGCC | I miR-s | GATCGCCCATGTAAATCGCGGCCTCTCTCT |
| AtPQL1&3 | (SEQ ID NO: 52) | | TTTGTATTCC |
| #1 | | | (SEQ ID NO: 26) |
| | | II miR-a | GAGGCCGCGATTTACATGGGCGATCAAAGA |
| | | | GAATCAATGA |
| | | | (SEQ ID NO: 27) |
| | | III miR*s | GAGGACGCGATTTACTTGGGCGTTCACAGG |
| | | | TCGTGATATG |
| | | | (SEQ ID NO: 28) |
| | | IV miR*a | GAACGCCCAAGTAAATCGCGTCCTCTACAT |
| | | | ATATATTCCT |
| | | | (SEQ ID NO: 29) |
| amiRNA | TCGCCCATGCTAAATGCCGGCC | I miR-s | GATCGCCCATGTAAATGCCGGCCTCTCTCT |
| AtPQL1&3 | (SEQ ID NO: 53) | | TTTGTATTCC |
| #2 | | | (SEQ ID NO: 30) |
| | | II miR-a | GAGGCCGGCATTTACATGGGCGATCAAAGA |
| | | | GAATCAATGA |
| | | | (SEQ ID NO: 31) |
| | | III miR*s | GAGGACGGCATTTACTTGGGCGTTCACAGG |
| | | | TCGTGATATG |
| | | | (SEQ ID NO: 32) |
| | | IV miR*a | GAACGCCCAAGTAAATGCCGTCCTCTACAT |
| | | | ATATATTCCT |
| | | | (SEQ ID NO: 33) |
| amiRNA | TATCAGTGGATTCAAACGCTC | I miR-s | GATATCAGTGGATTCAAACGCTCTCTCTCT |
| AtPQL2&3 | (SEQ ID NO: 54) | | TTTGTATTCC |
| #1 | | | (SEQ ID NO: 34) |
| | | II miR-a | GAGAGCGTTTGAATCCACTGATATCAAAGA |
| | | | GAATCAATGA |
| | | | (SEQ ID NO: 35) |
| | | III miR*s | GAGAACGTTTGAATCGACTGATTTCACAGG |
| | | | TCGTGATATG |
| | | | (SEQ ID NO: 36) |
| | | IV miR*a | GAAATCAGTCGATTCAAACGTTCTCTACAT |
| | | | ATATATTCCT |
| | | | (SEQ ID NO: 37) |
| amiRNA | TATCAATGGATTCAATACCTC | I miR-s | GATATCAATGGATTCAATACCTCTCTCTCT |
| AtPQL2&3 | (SEQ ID NO: 55) | | TTTGTATTCC |
| #2 | | | (SEQ ID NO: 38) |
| | | II miR-a | GAGAGGTATTGAATCCATTGATATCAAAGA |
| | | | GAATCAATGA |
| | | | (SEQ ID NO: 39) |
| | | III miR*s | GAGCGGTATTGAATGCATTGATATCACAGG |
| | | | TCGTGATATG |
| | | | (SEQ ID NO: 40) |
| | | IV miR*a | GATATCAATGCATTCAATACCGCTCTACAT |
| | | | ATATATTCCT |
| | | | (SEQ ID NO: 41) |
| amiRNA | TATCAATGGATTCAAACCCTC | I miR-s | GATATCAATGGATTCAAACCCTCTCTCTCT |
| AtPQL1, | (SEQ ID NO: 56) | | TTTGTATTCC |
| 2&3 #1 | | | (SEQ ID NO: 42) |
| | | II miR-a | GAGAGGGTTTGAATCCATTGATATCAAAGA |
| | | | GAATCAATGA |
| | | | (SEQ ID NO: 43) |
| | | III miR*s | GAGAAGGTTTGAATCGATTGATTTCACAGG |
| | | | TCGTGATATG |
| | | | (SEQ ID NO: 44) |
| | | IV miR*a | GAAATCAATCGATTCAAACCTTCTCTACAT |
| | | | ATATATTCCT |
| | | | (SEQ ID NO: 45) |
| amiRNA | TATCAATGGATTCAATCCTTC | I miR-s | GATATCAATGGATTCAATCCTTCTCTCTCT |
| AtPQL1, | (SEQ ID NO: 57) | | TTTGTATTCC |
| 2&3 #2 | | | (SEQ ID NO: 46) |
| | | II miR-a | GAGAAGGATTGAATCCATTGATATCAAAGA |
| | | | GAATCAATGA |
| | | | (SEQ ID NO: 47) |
| | | III miR*s | GAGACGGATTGAATCGATTGATTTCACAGG |
| | | | TCGTGATATG |
| | | | (SEQ ID NO: 48) |
| | | IV miR*a | GAAATCAATCGATTCAATCCGTCTCTACAT |
| | | | ATATATTCCT |
| | | | (SEQ ID NO: 49) |

### Arabidopsis Transformations

Arabidopsis Col-0 ecotype was transformed via the floral dip method (Clough & Bent, The Plant Journal 16: 735-743, 1998), using Agrobacterium tumefaciens, strain GV3101, with the pGWB2 or TOOL2 vectors containing either the 35S over-expression or amiRNA constructs. Seeds were collected from transformed plants and germinated on an artificial soil medium (3.6 L perlite-medium grade, 3.6 L coira and 0.25 L river sand) and sprayed with 100 mg L⁻¹ BASTA (AgrEvo, Dusseldorf, Germany) to identify putitative T₁ transformants. Transformants were transferred to soil, watered weekly with 300 ml of nutrient solution (2 mM Ca(NO₃), 15 mM KNO₃, 0.5 mM MgSO₄, 0.5 mM NaH₂PO₄, 15 mM NH₄NO₃, 2.5 μM NaFeEDTA, 200 μM H₃BO₃, 0.2 μM Na₂MoO₄, 0.2 μM NiCl₂, 1 μM ZnSO₄, 2 μM MnCl₂, 2 μM CuSO₄ and 0.2 μM CoCl₂) and grown to flowering to collect T₂ seed.

### Arabidopsis Salt Stress Assays

Seeds from mutant lines (35S, T-DNA KO, or amiRNA as described above) were surface sterilised, by soaking in 70% ethanol for two minutes followed by 5 rinses in sterile milli-Q water, before individual seeds were planted in 1.5 ml microfuge tube lids filled with Arabidopsis Germination Solution (Table 5) with 0.8% Bactoagar, pH 5.6. The lids were placed in germination trays sitting in Arabidopsis Germination Solution. The seeds were vernalised for 2 d at 4° C. and then transferred to a growth room with a 10 h light/14 h dark photoperiod, an irradiance of 150 mmol m⁻² s⁻¹, and a constant temperature of 21° C. After 2-3 weeks in germination trays, the plants were transferred to a constantly aerated hydroponics tank containing Arabidopsis Hydroponics Solution (Table 5). The pH of the hydroponic solution was monitored and maintained at pH 5.6. Salt stress was applied 1 week after placement in hydroponic tanks by the addition of 50 mM NaCl in 4 hourly increments of 25 mM. Calcium activity in the growth medium was maintained at 0.3 mM at each salt application by addition of the correct amount of calcium, as calculated using Visual Minteq Version 2.3 (US Environmental Protection Agency, USA).

Plants were harvested after 3 days of salt treatment. Whole shoots of control and salt treated plants were excised fresh weights recorded. The last fully expanded leaf was removed, weighed and digested in 1% nitric acid overnight at 85° C. in a Hot Block (Environmental Express, Mt Pleasant, S.C., USA). Na⁺ and K⁺ concentrations in this leaf were measured using a model 420 flame photometer (Sherwood, UK).

TABLE 5
Arabidopsis germination and hydroponic solutions

| Germination solution | | Hydroponic solution | |
|---|---|---|---|
| | Concentration | | Concentra- |
| Macronutrient | (mM) | Macronutrient | tion (mM) |
| CaCl2 | 0.75 | KNO3 | 1.25 |
| KCl | 1.0 | Ca(NO3)2•4H20 | 0.5 |
| Ca(NO3)2•4H20 | 0.25 | MgSO4•7H2O | 0.5 |
| MgSO4•7H20 | 1.0 | FeNaEDTA | 0.0425 |
| KH2PO4 | 0.2 | KH2PO4 | 0.0625 |
| | Concentration | | Concentra- |
| Micronutrient | (μM) | Micronutrient | tion (μM) |
| NaFe(III)EDTA | 50 | CuSO4•5H2O | 0.16 |
| H3BO3 | 50 | ZnSO4•7H2O | 0.38 |
| MnCl2•4H2O | 5 | MnSO4•H2O | 1.8 |
| ZnSO4•7H2O | 10 | H3BO3 | 45 |
| CuSO4•5H2O | 0.5 | (NH4)6Mo7O24•4H20 | 0.015 |
| Na2MoO3 | .01 | CoCl2•6H2) | 0.01 |

### Rice Transformation

Full length OsPQL1 was cloned from wild type Nipponbare rice plants into a pCR8 Gateway enabled entry vector. Restriction digestion and sequencing of the plasmid was used to confirm the orientation of the gene in the vector and to ensure there were no errors in the coding sequence of the cloned gene. For rice transformation, the gene was then transferred into a pMDC32 destination vector, using a Gateway reaction. The plasmid was sent to CIRAD, Montpellier, France, for rice transformation.

### Rice Salt Stress Assays

35S::OsPQL1 and wild type Nipponbare rice seeds were germinated for 5 days on moist filter paper at 28° C./25° C. day/night, 80%/60% day/night humidity and 600 μmol m⁻²s⁻¹ light, with a light dark cycle of 12 hrs light/12 hrs night. Seedlings were removed for the filter paper and placed in 1.5 ml microfuge tubes which had their bottoms removed to allow the roots to emerge from the tube. Each microfuge tube was placed carefully into a support above a 10 L tank filled with ACPFG rice nutrient solution (5 mM NH₄NO₃, 5.0 KNO₃, 2 mM Ca(NO₃)₂, 2.0 mM MgSO₄, 0.1 mM KH₂PO₄, 50 μM NaFemEDTA, 10 μM H₃BO₃, 5 μM MnCl₂, 5 μM ZnSO₄, 0.5 μM CuSO₄ and 0.1 μM Na₂MoO₃) allowing the seedling's root access to the media. Seedlings were grown for two weeks in 28° C./25° C. day/night, 80%/60% day/night humidity and 600 μmol m⁻²s⁻¹ light, with a light dark cycle of 12 hrs light/12 hrs night, with the nutrient solution replaced every 5 days. 14 days after germination, half of the seedlings were transferred into nutrient solution containing 75 mM NaCl, supplemented with 0.24 mM CaCl₂. So as not to shock the plants, salt application was made in three 12 hr applications of 25 mM NaCl and 0.8 mM CaCl₂. The plants were allowed to grow for a further 12 days before harvested. The 3^{rd} fully expanded leaf was removed from each plant, its fresh weight recorded and then incubated at 65° C. for 48 hrs to obtain dried tissue for dry weight measurements. Once weight measurements were obtained the tissue was digested for in 1% nitric acid overnight at 85° C. Na⁺ and K′ measurements for each leaf were determined by flame photometry.

### Yeast Transformation

Using primers AtPQL1, 2 or 3 Whole gene Forward and AtPQL1, 2 or 3 Whole gene Reverse (Table 3, supra), the complete gene was cloned from Arabidopsis genomic DNA into a pCR8 Gateway enabled entry vector. Restriction digestion and sequencing of the plasmid was used to confirm the orientation of the gene in the vector and to ensure there were no errors in the coding sequence of the cloned gene. Each gene was then transformed into S. cerevisiae using the lithium acetate/poly ethylene glycol method (Gietz et al., Yeast 11: 355-360, 1995) and transformants were selected on SD minimal media (0.67% (w.v) Difco Yeast nitrogen base without amino acids, 1 g/L Histidine), (-uracil). Transformed cells were individually grown overnight in SD minimal media (-uracil), 2% (w/v) D-galactose, to late log phase.

### Plate Assays

Cells were pelleted and re-suspended to an OD₆₀₀ of 0.3. Cultures were serially diluted to a final OD₆₀₀ of 0.0003 and 10 μl of each dilution was placed on SD minimal media (-uracil), 2% (w/v) D-galactose, 2% (w/v) agar, with either 0 mM NaCl+0 mM CaCl, 0 mM NaCl+10 mM CaCl, 500 mM NaCl+0 mM CaCl, or 500 mM NaCl+10 mM CaCl. Plates were incubated at 30° C. for 2-3 days and growth phenotypes were monitored.

### Liquid Culture Assays

Cells were added to 10 ml liquid cultures to reach a starting OD₆₀₀ of 0.1. Each 10 ml culture contained SD minimal media (-uracil), 2% (w/v) D-galactose, with either 0 mM NaCl+0 mM CaCl, 0 mM NaCl+10 mM CaCl, 500 mM NaCl+0 mM CaCl, or 500 mM NaCl+10 mM CaCl. 200 μl samples were taken every 24 hours for up to 4 days, to use for OD₆₀₀ measurements.

### Example 12

### PQ Loop Genes in Plants

Through genome database searches of Arabidopsis, rice and Physcomitrella patens, it is evident that homologues for the PQ loop genes (so named for two conserved pairs of proline (P) and glutamine (Q) amino acids found within each sequence) from yeast exist in all three genomes, particularly for the yeast gene, Yol092wp. Arabidopsis and Physcomitrella contain six PQ loop genes, while rice contains only three homologues. Intriguingly, these genes separate into three distinct clades (FIG. 18). The Arabidopsis genome contains three genes in clade I, two genes in clade II and a single gene in clade III. The rice genome contains a single gene in each clade. The Physcomitrella genome contains one gene in clade I, two in clade II, one in clade III and two that are more closely related to the second yeast gene (Ydr352wp), a group that is not found in higher plants. Thus, for discovery of viNSCC genes in higher plants, those most closely related to the yeast gene Yol092wp are most pertinent. The genes of clade I are the most closely related to Yol092wp and, therefore, are the most logical candidates.

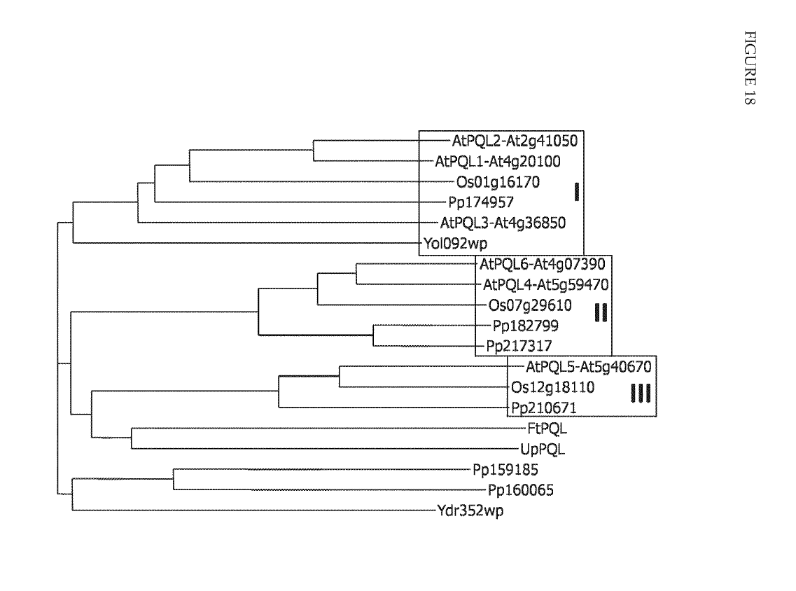

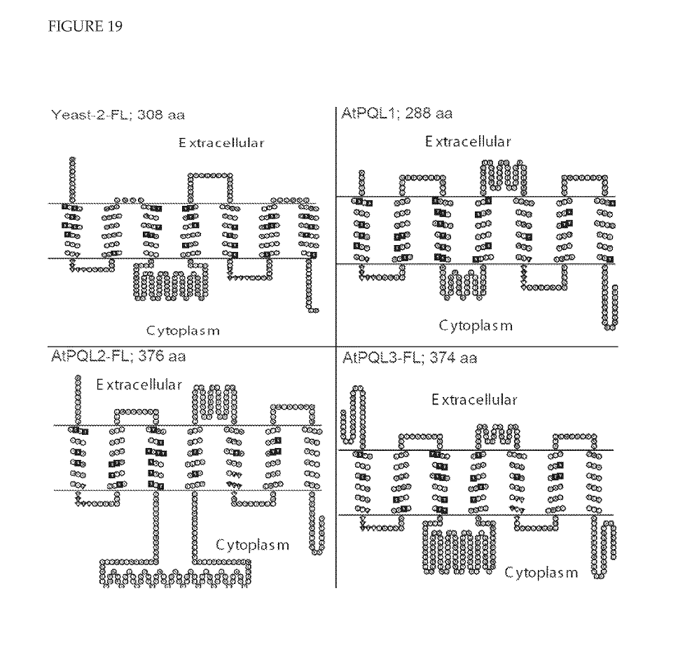

Additionally, topological analysis (using a consensus from several predictive programs including HMMTOP, PRED-TMR and a Kyte-Doolittle plot) of the putative protein structures of the PQ loop proteins from yeast and plants reveals much stronger similarity between the Yol092w and the plant members found in clade I, than with those in clades II and III. Clade I proteins have 7 transmembrane domains (TMD) and one large cytoplasmic loop of unknown function that connects TMDs 3 and 4 (FIG. 19). This contrasts with Clade II proteins, which have only 5 TMDs, and Clade III proteins which have only very small loops between the TMDs.

Since Arabidopsis contains three genes in clade I, there is potentially some level of redundancy in this clade, which indicates that analysis of multiple gene knockdowns and/or knockouts may be necessary to observe a phenotype when working to elucidate the function of the genes in this clade. Also, there is a likelihood that the proteins of this clade will assemble into multimers, and if these are heteromeric, phenotypic analysis may require multiple gene knockdowns.

PQL proteins are hypothesised to encode a non-selective cation channel, found on the plant cell's plasma membrane. These channels are thought to facilitate the influx of Na⁺ into cells. It is suspected to be expressed in root cells, possible in outer root cells. They are suspected to be involved in the initial influx of Na⁺ into plant roots.

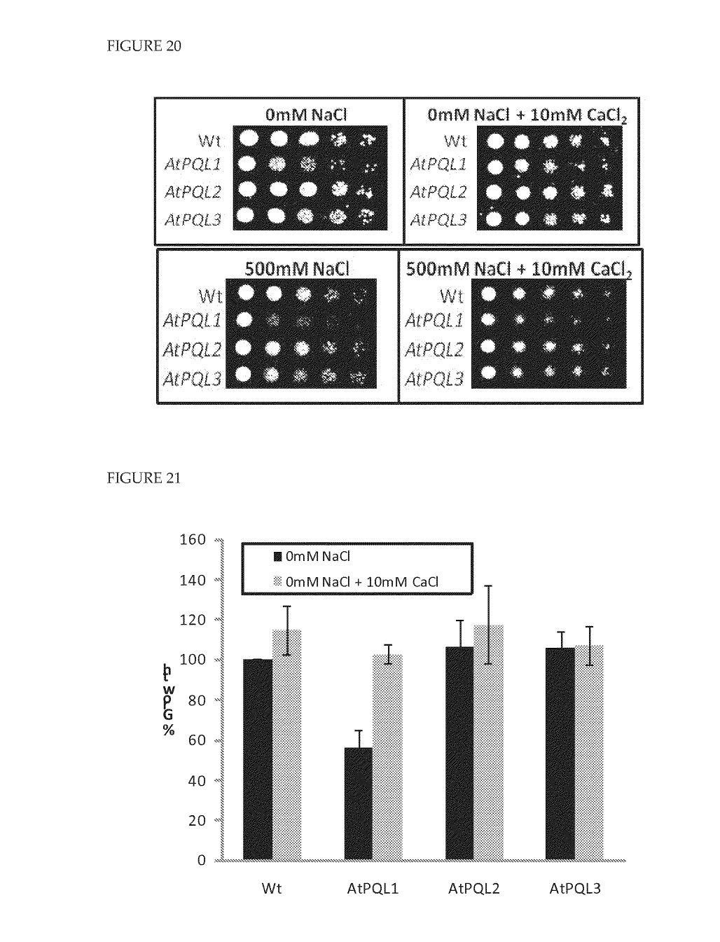

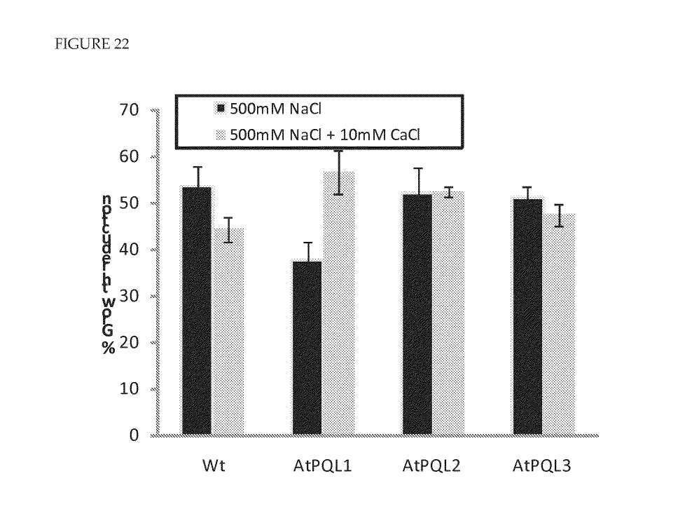

Yeast expressing the AtPQL1 gene demonstrate a slight growth reduction when grown on 0 mM NaCl when compared to yeast transformed with a vector control (wt) (FIGS. 20 and 21). Yeast expressing the AtPQL1 gene demonstrate significant growth reduction when grown on 500 mM NaCl, suggesting the gene encodes a protein that facilitates Na⁺ entry into the cell (FIGS. 20 and 22). This reduction in growth is significantly greater than in vector control yeast (wt). The effect is partially recovered by the addition of 10 mM CaCl₂ suggesting the protein involved is a non-selective cation channel (NSCC). NSCC activity can be inhibited by the addition of Ca²⁺ (FIGS. 20, 21 and 22).

As it is hypothesised that PQL proteins may be involved in the initial influx of Na⁺ to the root, and from there the Na⁺ is translocated to the shoot, amiRNA knockdowns and T-DNA knockouts of the AtPQL genes were obtained to investigate whether a reduction in the expression of these gene could reduce shoot Na⁺ accumulation. It was determined that both amiRNA knockdown of multiple AtPQL genes and T-DNA knockouts of individual AtPQL genes resulted in reduced shoot Na⁺ accumulation, suggesting they are indeed responsible for the initial influx of Na⁺ into the plant cell root.

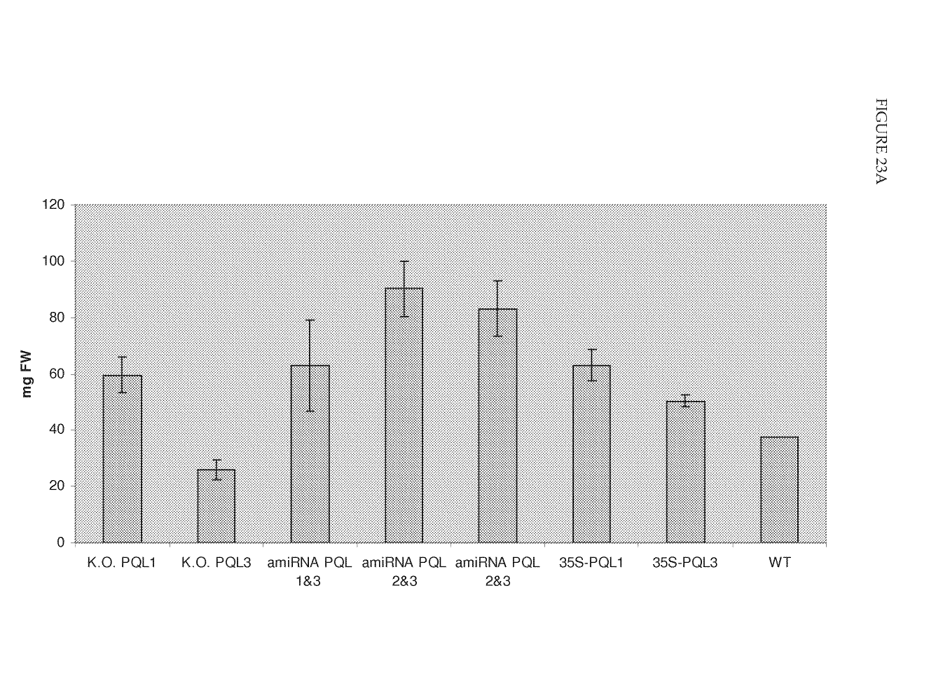

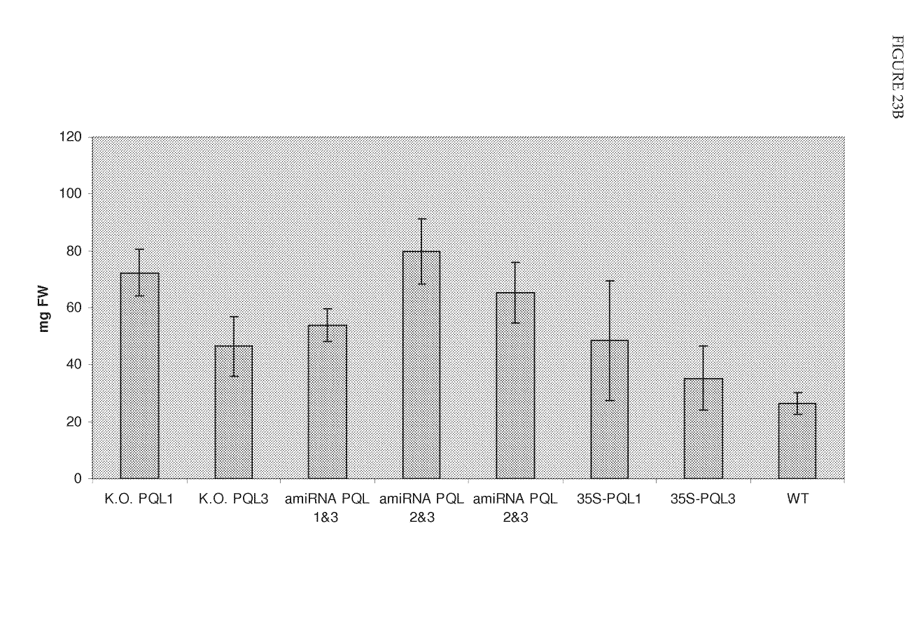

Under 0 mM NaCl, transgenic plants with individual PQL genes knocked out, multiple PQL genes knockdown or individual PQL genes over expressed, have slightly greater biomass that wild type control plants (FIG. 23A). The only exception appears to be the knockout of AtPQL3.

When the lines are exposed to 50 mM NaCl for 3 days all knockout, knockdown and overexpressing lines produce greater biomass than wild type controls (FIG. 23B). It was determined that both amiRNA knockdown of multiple AtPQL genes and T-DNA knockouts of individual AtPQL genes resulted in increased shoot biomass accumulation, suggesting they are indeed responsible for the initial influx of Na into the plant cell root and by reducing the expression of these genes the amount of Na⁺ into the plant is reduced, allowing the plant to put on more biomass (FIG. 23B). Interestingly, constitutive over-expression of AtPQL1 in every cell of an Arabidopsis plant also increased shoot biomass accumulation (FIG. 23B), again suggesting the protein encoded is indeed involved in Na⁺ transport. This apparently contradictory result can be attributed to the gene being expressed in every cell of the plant (where as the amiRNA knockdowns and T-DNA knockouts are reducing the native gene expression, which is suspected to be cell type and tissue specific). This constitutive expression has perhaps resulted in the expression of the PQL genes in beneficial cell types where the plant doesn't usually express the gene or has resulted in the plant upregulating some of its native Na⁺ transporters to compensate for the greater expression of the PQL genes. Similar results have been observed in the past with genes like AtHKT1; 1, where both T-DNA knockout plants and 35S::AtHKT1; 1 constitutive expression have increased shoot Na⁺ concentrations.

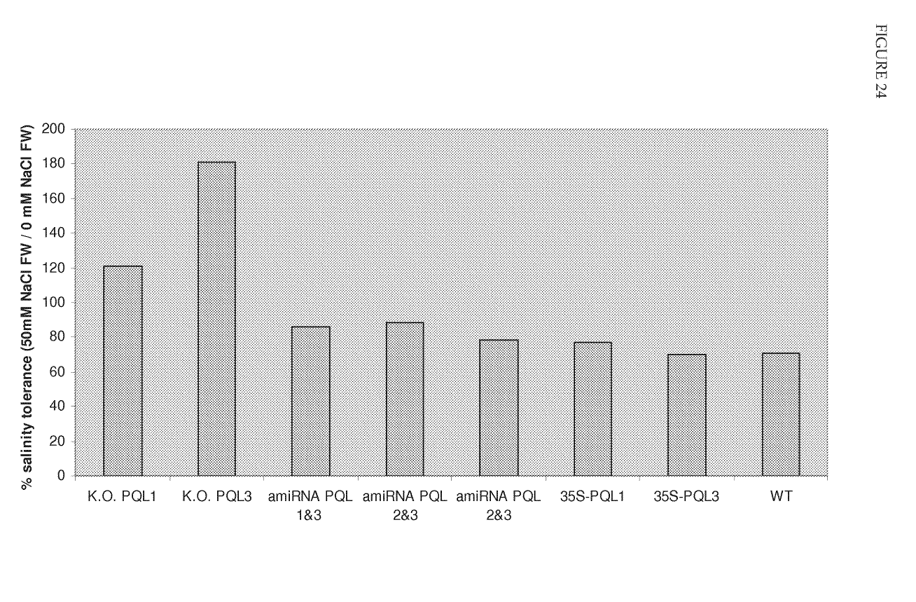

If the salinity tolerance of transgenic lines are calculated, by dividing the average biomass of the line under 50 mM salt stress by the average biomass of the line under no salt stress, it can be observed that knockout lines of AtPQL1 and AtPQL3 are highly salt tolerant when compared to wild type plants (120-180% salt tolerance in knockout lines as opposed to 70% in wild type plants). amiRNA knockdowns, while not being as salt tolerant as complete knockouts, are still more salt tolerant that wild type plants (80-85% salt tolerance in amiRNA lines as opposed to 70% salt tolerance in wild type plants). Lines constitutively expressing AtPQL genes are similar in salinity tolerance to wildtype plants (FIG. 24).

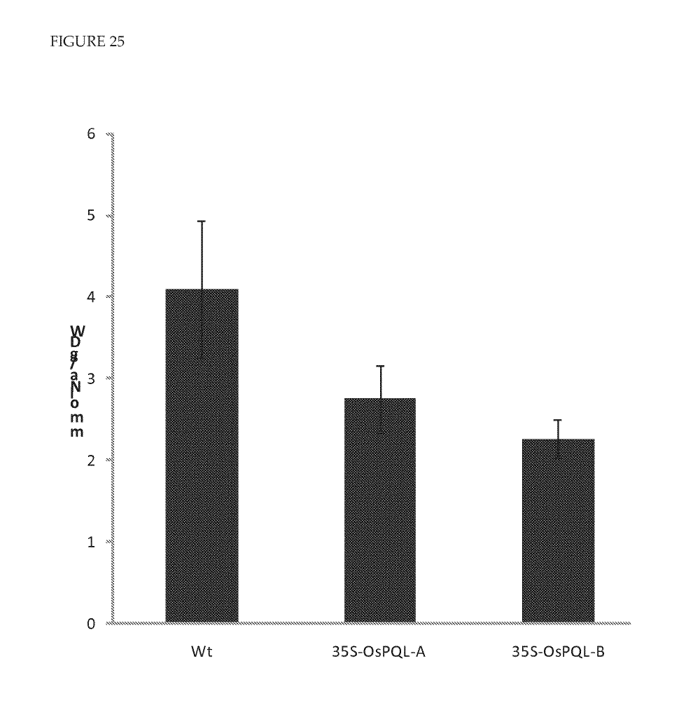

As with Arabidopsis, constitutive over-expression of OsPQL1 in every cell of a rice plant also significantly reduced shoot Na accumulation (FIG. 25), suggesting the rice protein encoded is also involved in Na⁺ transport.

### Example 13

### Localisation of AtPQL1 in Tobacco Epidermal Cells

AtPQL1-GFP fusions were transiently expressed in tobacco epidermal cells using the agroinfiltration method as described by Tang et al. (Science 274: 2060-2063, 1996). Infiltrated plants were returned to the growth room for 3 days before observation. Infiltrated areas of the leaf were then collected by excising an area of leaf (approx 1 cm²) with a razor blade. To reduce background fluorescence due to air pockets, excised leaf samples were vacuum infiltrated with distilled water. A Zeiss CLSM510-UV microscope with a x20 Plan Apochromat objective was used to view leaf discs. GFP-fluorescence was excited at 488 nm with an argon laser. An NFT545 dichroic filter was used to split the emitted fluorescent light between two channels, with a 505-530 nm band-pass filter for GFP and a 560-615 nm band pass filter for chloroplast autofluorescence.

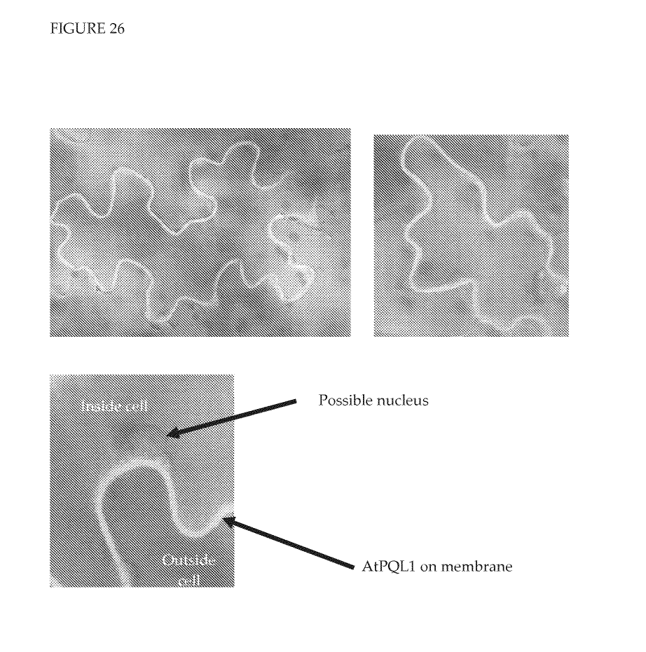

As shown in FIG. 26, visualisation of GFP fluorescence by confocal microscopy suggests the AtPQL1 protein is localised to a membrane in the cell. Due to the location of cellular organelles, it is suspected that AtPQL1 is localised on the plasma membrane.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of the steps or features.

Also, it must be noted that, as used herein, the singular forms “a”, “an” and “the” include plural aspects unless the context already dictates otherwise.

1. A method for modulating the rate, level or pattern of cation flux across a cell membrane, the method comprising modulating the expression of a PQ-loop repeat polypeptide in a cell comprising the cell membrane.

2. The method according to claim 1, wherein the PQ-loop repeat polypeptide comprises a Voltage insensitive Non-Selective Cation Channel.

3. The method according to claim 1, wherein the PQ-loop repeat polypeptide comprises a monovalent cation transporter.

4. The method according to claim 3, wherein the monovalent cation comprises one or more of Na⁺, K⁺, NH₄⁺, methylammonium, Tris⁺ or choline⁺.

5. The method according to claim 3, wherein monovalent cation transport by the polypeptide may be inhibited by a polyvalent cation.

6. The method according to claim 5, wherein the polyvalent cation is Ca²⁺.

7. The method according to claim 1 wherein the PQ-loop repeat polypeptide comprises a YOL092w-like polypeptide or a YDR352w-like polypeptide.

8-9. (canceled)

10. The method according to claim 1, wherein modulating the rate, level or pattern of cation flux across a cell membrane of the cell modulates the cation tolerance or sensitivity of the cell.

11-12. (canceled)

13. The method according to claim 1, wherein the expression of the polypeptide is modulated by modulating the expression of a nucleic acid which encodes a PQ loop repeat polypeptide in the cell.

14. A cell with a modulated rate, level or pattern of cation flux across a membrane of the cell, wherein said modulation is the result of modulated expression of a PQ-loop repeat polypeptide in the cell.

15. The cell according to claim 14, wherein the rate, level or pattern of cation flux across a membrane of the cell is modulated by modulating expression of a PQ-loop repeat polypeptide in the cell.

16. A multicellular structure comprising one or more cells according to claim 14.

17. A nucleic acid construct comprising a PQ loop repeat polypeptide encoding nucleic acid.

18. The nucleic acid construct according to claim 17, wherein the PQ-loop repeat polypeptide comprises a Voltage insensitive Non-Selective Cation Channel.

19. The nucleic acid construct according to claim 17, wherein the PQ-loop repeat polypeptide comprises a monovalent cation transporter.

20. (canceled)

21. The nucleic acid construct according to claim 19, wherein monovalent cation transport by the polypeptide may be inhibited by a polyvalent cation.

22. (canceled)

23. The nucleic acid construct according to claim 17 wherein the PQ-loop repeat polypeptide comprises a YOL092w-like polypeptide or a YDR352w-like polypeptide.

24-26. (canceled)

27. A method for ascertaining the cation sensitivity or tolerance of an organism, the method comprising determining the expression of a PQ loop repeat polypeptide encoding nucleic acid in one or more cells of the organism.

28. The method according to claim 27, wherein the PQ-loop repeat polypeptide comprises a voltage insensitive Non-Selective Cation Channel.

29. The method according to claim 27, wherein the PQ-loop repeat polypeptide comprises a monovalent cation transporter.

30. (canceled)

31. The method according to claim 29, wherein monovalent cation transport by the polypeptide may be inhibited by a polyvalent cation.

32. (canceled)

33. The method according to claim 27 wherein the PQ-loop repeat polypeptide comprises a YOL092w-like polypeptide or a YDR352w-like polypeptide.

34. (canceled)


SEQUENCE LISTING (57 sequences)

SEQ ID NO: 1 - protein, 8 aa, Artificial sequence
  amino acid motif
RLPQIXXN

SEQ ID NO: 2 - protein, 4 aa, Artificial sequence
  amino acid motif
YXLS

SEQ ID NO: 3 - protein, 353 aa, Artificial sequence
  YDR352w-like consensus sequence
MPDCCNDTACVSVTATVLARVVTRWSHGPLVRRSLDSLYTRCPRFLAGPALLPGIMVWKKLGSRNFSSCPGSIQWIWDVLGCDGWDSLGLISILAASFPQFIKYKTDALSWFLLWGGDNLIGLDQLQTTAYVLADMLTLYFYYKPSFPEKEPRHSTEKDPGMTEGKLDVVYRLVSPPSGLSGRGKKFRTRPSLLSAPINSVLLLGSPPDLTASTSPSLNRELRARLLGKGPLRKHRKYVGIAAIASVVCLGATALSLGSGNQSAVSVVGSSLPPIGFVIGISSVLYLLSRLPQINFLRKSTGISLFALGNYGLSGQSEGSYLLHHLPWLVGSLGVLLLDIQYREPLLPSLLIK

SEQ ID NO: 4 - protein, 317 aa, Saccharomyces cerevisiae
MSCSNGIWPTVSNLCGSLSFFTSVISLFPQIIETYRDKSVDGLSPYFLLAWLCGDITSLIGAKLTGQLLFQILLAIYFLLNDSFVCGQYYYYGVLHENKLATVGHEPKPLLPELVENGELLREEEDMIQGGSSAESPRSSRRRSAITAALAIAHTISTASAYPLNVGSTQSQVGPPGDGKNSQLGTILSWIGASFYVGARIPQLIKNYNRKSTDGLSPFLFATTLLCNITYNLSIFTSCRFLDNQNKREFIVNELPFIFGSAGTIAFDLIYFYQYYILYATDMQLRELERELYSPEEDSAAQLVTERTSLLSGETQT

SEQ ID NO: 5 - protein, 7 aa, Artificial sequence
  amino acid motif
PQIXXNF

SEQ ID NO: 6 - protein, 6 aa, Artificial sequence
  amino acid motif
GDIFNL

SEQ ID NO: 7 - protein, 9 aa, Artificial sequence
  amino acid motif
PQIXLNXKR

SEQ ID NO: 8 - protein, 198 aa, Artificial sequence
  YOL092w-like consensus sequence
MGIFSGMVSLKCRWVIDLSLGSWVAEPQITNFKSEGLSLFLWGDIFNLGCEPALPTQFYALYTVTILQYYDKRKPKGPKVIGEIRNNSHTIPGDSSREYYTAREGSYSCKFLSRVFVVRARKLSGLGWMAIYMGGRIPQILNKRGSEGLNPLMFFALGNTYVSILVDSWPNLPWLDAGCVLDIQFYKEVKTFVSEDSV

SEQ ID NO: 9 - protein, 308 aa, Saccharomyces cerevisiae
MQLVPLELNRSTLSGISGSISISCWIIVFVPQIYENFYRKSSDGLSLLFVVLWLAGDVFNLMGAVMQHLLSTMIILAAYYTVADIILLGQCLWYDNEEKPAVDPIHLSPANPINENVLHDVFNEQQPLLNSQGQPNRIDEEMAAPSSDGNAGDDNLREVNSRNLIKDIFIVSGVVFVGFISWYVTYCVNYTQPPPVEDPSLPVPELQINWMAQIFGYLSALLYLGSRIPQILLNFKRKSCEGISFLFFLFACLGNTTFIFSVIVISLDWKYLIMNASWLVGSIGTLFMDFVIFSQFFIYKRNKKFILN

SEQ ID NO: 10 - DNA, 20 bp, Artificial Sequence
  primer
atgattcgagatgatttgtc

SEQ ID NO: 11 - DNA, 20 bp, Artificial Sequence
  primer
ttagacagcttcttcaccgg

SEQ ID NO: 12 - DNA, 20 bp, Artificial Sequence
  primer
atgtttctccacagcagttt

SEQ ID NO: 13 - DNA, 19 bp, Artificial Sequence
  primer
ttagacagcttctgcggtt

SEQ ID NO: 14 - DNA, 20 bp, Artificial Sequence
  primer
atggattatctaggaatcga

SEQ ID NO: 15 - DNA, 20 bp, Artificial Sequence
  primer
tgaaatctttttacttttca

SEQ ID NO: 16 - DNA, 20 bp, Artificial Sequence
  primer
atgggcatcttcagtggagc

SEQ ID NO: 17 - DNA, 21 bp, Artificial Sequence
  primer
ttaaactttatcagcactgtc

SEQ ID NO: 18 - DNA, 40 bp, Artificial Sequence
  primer
gatattaatggattctatccctctctctcttttgtattcc

SEQ ID NO: 19 - DNA, 40 bp, Artificial Sequence
  primer
gagagggatagaatccattaatatcaaagagaatcaatga

SEQ ID NO: 20 - DNA, 40 bp, Artificial Sequence
  primer
gagaaggatagaatcgattaatttcacaggtcgtgatatg

SEQ ID NO: 21 - DNA, 40 bp, Artificial Sequence
  primer
gaaattaatcgattctatccttctctacatatatattcct

SEQ ID NO: 22 - DNA, 40 bp, Artificial Sequence
  primer
gatagttagtcataatctacggttctctcttttgtattcc

SEQ ID NO: 23 - DNA, 40 bp, Artificial Sequence
  primer
gaaccgtagattatgactaactatcaaagagaatcaatga

SEQ ID NO: 24 - DNA, 40 bp, Artificial Sequence
  primer
gaacagtagattatgtctaactttcacaggtcgtgatatg

SEQ ID NO: 25 - DNA, 40 bp, Artificial Sequence
  primer
gaaagttagacataatctactgttctacatatatattcct

SEQ ID NO: 26 - DNA, 40 bp, Artificial Sequence
  primer
gatcgcccatgtaaatcgcggcctctctcttttgtattcc

SEQ ID NO: 27 - DNA, 40 bp, Artificial Sequence
  primer
gaggccgcgatttacatgggcgatcaaagagaatcaatga

SEQ ID NO: 28 - DNA, 40 bp, Artificial Sequence
  primer
gaggacgcgatttacttgggcgttcacaggtcgtgatatg

SEQ ID NO: 29 - DNA, 40 bp, Artificial Sequence
  primer
gaacgcccaagtaaatcgcgtcctctacatatatattcct

SEQ ID NO: 30 - DNA, 40 bp, Artificial Sequence
  primer
gatcgcccatgtaaatgccggcctctctcttttgtattcc

SEQ ID NO: 31 - DNA, 40 bp, Artificial Sequence
  primer
gaggccggcatttacatgggcgatcaaagagaatcaatga

SEQ ID NO: 32 - DNA, 40 bp, Artificial Sequence
  primer
gaggacggcatttacttgggcgttcacaggtcgtgatatg

SEQ ID NO: 33 - DNA, 40 bp, Artificial Sequence
  primer
gaacgcccaagtaaatgccgtcctctacatatatattcct

SEQ ID NO: 34 - DNA, 40 bp, Artificial Sequence
  primer
gatatcagtggattcaaacgctctctctcttttgtattcc

SEQ ID NO: 35 - DNA, 40 bp, Artificial Sequence
  primer
gagagcgtttgaatccactgatatcaaagagaatcaatga

SEQ ID NO: 36 - DNA, 40 bp, Artificial Sequence
  primer
gagaacgtttgaatcgactgatttcacaggtcgtgatatg

SEQ ID NO: 37 - DNA, 40 bp, Artificial Sequence
  primer
gaaatcagtcgattcaaacgttctctacatatatattcct

SEQ ID NO: 38 - DNA, 40 bp, Artificial Sequence
  primer
gatatcaatggattcaatacctctctctcttttgtattcc

SEQ ID NO: 39 - DNA, 40 bp, Artificial Sequence
  primer
gagaggtattgaatccattgatatcaaagagaatcaatga

SEQ ID NO: 40 - DNA, 40 bp, Artificial Sequence
  primer
gagcggtattgaatgcattgatatcacaggtcgtgatatg

SEQ ID NO: 41 - DNA, 40 bp, Artificial Sequence
  primer
gatatcaatgcattcaataccgctctacatatatattcct

SEQ ID NO: 42 - DNA, 40 bp, Artificial Sequence
  primer
gatatcaatggattcaaaccctctctctcttttgtattcc

SEQ ID NO: 43 - DNA, 40 bp, Artificial Sequence
  primer
gagagggtttgaatccattgatatcaaagagaatcaatga

SEQ ID NO: 44 - DNA, 40 bp, Artificial Sequence
  primer
gagaaggtttgaatcgattgatttcacaggtcgtgatatg

SEQ ID NO: 45 - DNA, 40 bp, Artificial Sequence
  primer
gaaatcaatcgattcaaaccttctctacatatatattcct

SEQ ID NO: 46 - DNA, 40 bp, Artificial Sequence
  primer
gatatcaatggattcaatccttctctctcttttgtattcc

SEQ ID NO: 47 - DNA, 40 bp, Artificial Sequence
  primer
gagaaggattgaatccattgatatcaaagagaatcaatga

SEQ ID NO: 48 - DNA, 40 bp, Artificial Sequence
  primer
gagacggattgaatcgattgatttcacaggtcgtgatatg

SEQ ID NO: 49 - DNA, 40 bp, Artificial Sequence
  primer
gaaatcaatcgattcaatccgtctctacatatatattcct

SEQ ID NO: 50 - DNA, 21 bp, Artificial Sequence
  amiRNA target sequence
tattaatggattctatccctc

SEQ ID NO: 51 - DNA, 21 bp, Artificial Sequence
  amiRNA target sequence
tagttagtcataatctacggt

SEQ ID NO: 52 - DNA, 21 bp, Artificial Sequence
  amiRNA target sequence
tcgcccatgtaaatcgcggcc

SEQ ID NO: 53 - DNA, 22 bp, Artificial Sequence
  amiRNA target sequence
tcgcccatgctaaatgccggcc

SEQ ID NO: 54 - DNA, 21 bp, Artificial Sequence
  amiRNA target sequence
tatcagtggattcaaacgctc

SEQ ID NO: 55 - DNA, 21 bp, Artificial Sequence
  amiRNA target sequence
tatcaatggattcaatacctc

SEQ ID NO: 56 - DNA, 21 bp, Artificial Sequence
  amiRNA target sequence
tatcaatggattcaaaccctc

SEQ ID NO: 57 - DNA, 21 bp, Artificial Sequence
  amiRNA target sequence
tatcaatggattcaatccttc